SEQUENCE LISTING (26 sequences)

SEQ ID NO: 1 - DNA, 762 bp, homo sapiens
atgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccag

SEQ ID NO: 2 - DNA, 705 bp, homo sapiens
gatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - DNA, 1467 bp, homo sapiens
atgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccaggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 254 aa, homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQ

SEQ ID NO: 5 - protein, 234 aa, homo sapiens
DPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 488 aa, homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 570 bp, homo sapiens
atggcctggcgagccctacacccactgctactgctgctgctgctgttcccaggctctcaggcacaatccaaggctcaggtacttcaaagtgtggcagggcagacgctaaccgtgagatgccagtacccgcccacgggcagtctctacgagaagaaaggctggtgtaaggaggcttcagcacttgtgtgcatcaggttagtcaccagctccaagcccaggacgatggcttggacctctcgattcacaatctgggacgaccctgatgctggcttcttcactgtcaccatgactgatctgagagaggaagactcaggacattactggtgtagaatctaccgcccttctgacaactctgtctctaagtccgtcagattctatctggtggtatctccagcctctgcctccacacagaccccctggactccccgcgacctggtctcttcacagacccagacccagagctgtgtgcctcccactgcaggagccagacaagcccctgagtctccatctaccatccctgtcccttcacagccacagaactccacgctccgccctggccctgcagccccc

SEQ ID NO: 8 - DNA, 1275 bp, homo sapiens
atggcctggcgagccctacacccactgctactgctgctgctgctgttcccaggctctcaggcacaatccaaggctcaggtacttcaaagtgtggcagggcagacgctaaccgtgagatgccagtacccgcccacgggcagtctctacgagaagaaaggctggtgtaaggaggcttcagcacttgtgtgcatcaggttagtcaccagctccaagcccaggacgatggcttggacctctcgattcacaatctgggacgaccctgatgctggcttcttcactgtcaccatgactgatctgagagaggaagactcaggacattactggtgtagaatctaccgcccttctgacaactctgtctctaagtccgtcagattctatctggtggtatctccagcctctgcctccacacagaccccctggactccccgcgacctggtctcttcacagacccagacccagagctgtgtgcctcccactgcaggagccagacaagcccctgagtctccatctaccatccctgtcccttcacagccacagaactccacgctccgccctggccctgcagcccccgatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - protein, 190 aa, homo sapiens
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTPWTPRDLVSSQTQTQSCVPPTAGARQAPESPSTIPVPSQPQNSTLRPGPAAP

SEQ ID NO: 10 - protein, 424 aa, homo sapiens
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTPWTPRDLVSSQTQTQSCVPPTAGARQAPESPSTIPVPSQPQNSTLRPGPAAPDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 711 bp, homo sapiens
atgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgaccatgccctctgggatcacactgcccag

SEQ ID NO: 12 - DNA, 1416 bp, homo sapiens
atgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgaccatgccctctgggatcacactgcccaggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 13 - protein, 237 aa, homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDHALWDHTAQ

SEQ ID NO: 14 - protein, 471 aa, homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDHALWDHTAQDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 405 bp, homo sapiens
atggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggg

SEQ ID NO: 16 - DNA, 1110 bp, homo sapiens
atggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 17 - protein, 135 aa, homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLG

SEQ ID NO: 18 - protein, 369 aa, homo sapiens [sequence omitted]

SEQ ID NO: 19 - DNA, 402 bp, homo sapiens
tatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccag

SEQ ID NO: 20 - DNA, 351 bp, homo sapiens
tatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgaccatgccctctgggatcacactgcccag

SEQ ID NO: 21 - DNA, 240 bp, homo sapiens
atctaccgcccttctgacaactctgtctctaagtccgtcagattctatctggtggtatctccagcctctgcctccacacagaccccctggactccccgcgacctggtctcttcacagacccagacccagagctgtgtgcctcccactgcaggagccagacaagcccctgagtctccatctaccatccctgtcccttcacagccacagaactccacgctccgccctggccctgcagccccc

SEQ ID NO: 22 - protein, 134 aa, homo sapiens
YDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQ

SEQ ID NO: 23 - protein, 117 aa, homo sapiens
YDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDHALWDHTAQ

SEQ ID NO: 24 - protein, 79 aa, homo sapiens
IYRPSDNSVSKSVRFYLVVSPASASTQTPWTPRDLVSSQTQTQSCVPPTAGARQAPESPSTIPVPSQPQNSTLRPGPAP

SEQ ID NO: 25 - DNA, 360 bp, homo sapiens
atgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatg

SEQ ID NO: 26 - protein, 120 aa, homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEM

(19) United States
(12) Patent Application Publication
(10) Pub. No.: US 2004/0072256 A1
(43) Pub. Date: Apr. 15, 2004

(54) Nk cells activiating receptors and their therapeutic and diagnostic uses

(76) Inventors: Ofer Mandelboim, Shoam (IL); Angel Porgador, Lehavim (IL)

Correspondence Address: Cooper & Dunham, 1185 Avenue of the Americas, New York, NY, 10036, US

(21) Appl. No.: 10/333,481

(22) PCT Filed: Jul. 19, 2001
(86) PCT No.: PCT/IL01/00664

(30) Foreign Application Priority Data
Jul. 20, 2000 (IL) 137419

(51) Int. Cl. G01N 33/53; G01N 33/567; C07K 16/46

(52) U.S. Cl. 435/7.2; 530/391.1

(57) ABSTRACT

The invention relates to a targeting complex, capable of targeting an active substance to a target cell, said complex comprising: a target recognition segment comprising one of NKp46, NKp30, NKp44 or a functional fragment thereof; and an active segment comprising an active substance such as cytotoxic moiety; an imaging moiety; or an Ig fragment. The targeting complex of the invention serves as a therapeutic agents for the treatment of pathologies associated with viral infections or cancer and for the imaging and monitoring of cancer.

### FIELD OF THE INVENTION

The invention relates to therapeutic agents for the treatment of pathologies associated with viral infections or cancer and for the imaging and monitoring of cancer. More particularly, the present invention provides compositions and methods for the treatment and detection of a variety of viral infections, by using complex agents comprising the NK cells activating proteins, NKp46 and NKp44, and functional fragments thereof, linked to therapeutic or imaging agents.

### BACKGROUND OF THE INVENTION

Natural Killer (NK) cells, as well as cytotoxic T lymphocytes (CTL), are major components of the cellular mechanism by which an immune response leads to the destruction of foreign or infected tissue [Trinchieri, et al., Adv. in Immunol. 47:187-376 (1989)]. In contrast to CTL, which are activated in the presence of class I MHC molecules and an appropriate specific peptide, one well defined function of NK cells is the lysis of target cells deficient in expression of MHC class I proteins. In this manner NK cells carry out immuno-surveillance for “miself” [Ljunggren et al., Immunol. Today 11:7-10 (1990)], rather than for direct detection of foreign antigens.

Thus, NK cells, generally representing about 10-15% of circulating lymphocytes, bind and kill target cells including virus-infected cells and many malignant cells, in a nonspecific manner with regard to antigen and without prior immune sensitization [Herberman et al., Science 214:24-27 (1981)].

Recognition of polymorphic determinants on HLA molecules by human NK cell inhibitory receptors is mediated by three types of class I MHC-binding receptors: the Ig superfamily of inhibitory receptors which includes both the NKIR proteins [Colonna, et al., Science 268:405-408 (1995); Wagtmann, et al., Immunity 2:439-449 (1995); D'Andre, et al., J. Immunol 155:2306-2310 (1995)] and the ILT-2 protein [Colonna, et al., J. Exp. Med. 186:1809-1818 (1997)], whose ligands are various HLA-A, -B and -C proteins, and the mucin-like CD94/NKG2 complex, which delivers an inhibitory signal upon binding the HLA-E protein [Borrego et al., J. Exp. Med. 187:813-818 (1998); Brnud et al., Nature, 391:795-799 (1998); Lee et al., Proc. Natl. Acad. Sci. USA, 95:5199-5204 (1998)]. This variety of class I MHC protein-specific receptors illustrates the importance of these molecules in modulating NK function.

However, lysis receptor(s) involved in triggering NK cell cytotoxicity against target cells are little understood. Four candidate lysis receptors were recently identified, NKp30, NKp44 [Cantoni, C. et al., J. Exp. Med. 189:787-796 (1999)], NKp46 [Pessino et al., J. Exp. Med. 188:953-960 (1998)] and CD16 [Mandelboim et al., Proc. Natl. Acad. Sci. USA. 96:5640-5644 (1999)]. The NKp46 receptor is conceded to be the major lysis receptor involved in killing target cells as it is expressed on all NK cells, whether activated or non-activated [Pessino et al., (1998) ibid.]. The present invention is based on some identification and characterization of “lysis ligand(s)” for NKp44 and NKp46. With the exception of NKp44, all of these receptors are expressed on the surface of both activated and non activated NK cells and all transduce activation signals via association with CD3ζ/FcεRIγ [Bottino, C., et al., Hum. Immunol. 61: 1-6 (2000). In contrast, the NKp44 receptor is expressed on the surface of activating NK cells only and delivers its activating signal via the association with DAP12 [Lanier L. L., et al., Nature. 391: 703-707 (1998)]. The lysis ligands that are recognized by these receptors are unknown.

The findings of the present inventors show that soluble NKp44- and NKp46-Ig fusion proteins bind to the hemagglutinin (HA) of Influenza virus, and the hemagglutinin-neuraminidase (HN) of parainfluenza virus, and that binding of NKp44 and NKp46 to these viral proteins is required for lysis of cells expressing the corresponding glycoproteins. The binding requires the sialylation of NKp44 and NKp46 oligosaccharides, which is consistent with the known sialic binding capacity of the viral glycoproteins. These findings explain how NKp44- and NKp46-expressing cells can recognize Influenza and parainfluenza virus-infected target cells without a major decrease in target cell class I molecule expression. As sialic acid is utilized as a receptor for a number of other viruses, a general strategy for NK recognition of a substantial subset of viral pathogens may be suggested.

### SUMMARY OF THE INVENTION

In a first aspect, the present invention relates to a targeting complex, capable of targeting an active substance to a target cell. This complex comprises a target recognition segment, which comprises at least NKp46, NKp30, NKp44 or a functional fragment thereof, and an active segment comprising the active substance which may be a cytotoxic moiety, an imaging moiety or an Ig fragment.

The target recognition segment is derived from NKp46 and preferably comprises at least one of domains 1 and 2 of the NKp46 molecule, more preferably both domains 1 and 2. In another particularly preferred embodiment the segment comprises domain 2 of the NKp46 molecule. Alternatively, the target recognition segment is derived from NKp44 and preferably comprises at least one of domains 1 and 2 of the NKp44 molecule, more preferably both domains 1 and 2. In another particularly preferred embodiment the segment comprises domain 2 of the NKp44 molecule.

In a specifically preferred embodiment the complex the invention is a fusion protein comprising as the active segment an Ig fragment. This Ig fragment is preferably the Fc portion of an Ig molecule.

Alternatively, the complex of the present invention is a conjugate comprising as an active segment a cytotoxic moiety. This cytotoxic moiety may be a cytotoxin or an anticellular agent, which is capable of killing and/or suppressing the growth or cell division of the target cell.

Preferred conjugates of the invention may comprise as the cytotoxin or anticellular agent a synthetic toxin or a toxin derived from plants, fungi, or bacteria. More specifically, this toxin can be selected from any one of A chain toxin, ribosome inactivating protein, α-sarcin, aspergillin, restrictocin, ribonuclease, diphtheria toxin, Pseudomonas exotoxin, an endotoxin or the lipid A moiety of an endotoxin.

In yet another alternative embodiment, the complex of the present invention may be a conjugate in which the active segment is an imaging moiety. The imaging moiety may be any detectable label, such as paramagnetic, radioactive and fluorogenic labels.

In a specific embodiment, the complex of the present invention may be specifically targeted to cells derived from solid as well as non-solid tumors, particularly malignant tumors.

Alternatively, the complex of the present invention is targeted to a defective or diseased cell. Such target cells may be pathogenic virus-infected cells. More specifically, the pathogenic virus may be any of variety of viruses including, but not limited to, Influenza virus, human immunodeficiency virus, Epstein-Barr virus, cytomegalovirus, Vaccinia virus and Herpes virus.

Preferably, where the target cell is a virus-infected cell, the complex of the invention has a target recognition segment which is capable of binding to a ligand expressed on the surface of said target cell, this binding being mediated by sialic acid.

A second aspect the present invention relates to an expression vector comprising DNA coding for a NKp46-Ig fusion protein. This DNA comprises a segment encoding NKp46 or functional fragments thereof, preferably a NKp46 fragment comprising at least one of domains 1 and 2 of the NKp46 molecule, more preferably both domains 1 and 2 of the NKp46 molecule. This expression vector further comprises a second segment comprises a DNA sequence encoding the Fc portion of an Ig molecule. In another preferred embodiment, the first segment may comprise the nucleic acid sequence of domain 2 alone.

In another embodiment of the present aspect, the invention relates to an expression vector comprising DNA coding for a NKp44-Ig fusion protein. This DNA comprises a segment encoding NKp44 or functional fragments thereof, preferably a NKp44 fragment comprising at least one of domains 1 and 2 of the NKp44 molecule, more preferably both domains 1 and 2. In another preferred embodiment, the first segment may comprise the nucleic acid sequence of domain 2 alone. The second segment of the DNA comprised in the expression vector of the invention comprises a DNA sequence encoding the Fc portion of an Ig molecule.

The invention also relates to a host cell transformed with the DNA or expression vectors of the invention.

A specifically preferred embodiment of the invention relates to a NKp46-Ig fusion protein. This fusion protein comprises the amino acid sequence substantially as denoted by any one of SEQ ID NO: 6 and NO: 14, and is encoded by the nucleic acid sequence substantially as denoted by any one of SEQ ID NO: 3 and NO: 12.

In yet another specifically preferred embodiment the invention relates to a NKp44-Ig fusion protein. This fusion protein comprises the amino acid sequence substantially as denoted by SEQ ID NO: 10, and is encoded by the nucleic acid sequence substantially as denoted by SEQ ID NO: 8.

Another aspect of the present invention relates to antibodies that specifically recognize and bind to the fusion proteins NKp46-Ig or to NKp44-Ig of the invention.

Additionally, the invention relates to an antibody that specifically recognizes and binds to an epitope on a protein, which protein is a ligand for the NK cell activating receptor NKp46 or NKp44. A specifically preferred antibody is the antibody designated as 135.7. The antibodies of the invention may be mono- or polyclonal antibodies. Further, the antibodies of the invention may be conjugated to a detectable moiety.

In a further aspect the invention relates to compositions for the treatment of pathological conditions. The compositions of the invention comprise as active ingredient a complex comprising a target recognition segment and an active segment. The target recognition segment is capable of specifically recognizing and binding to a diseased target cell involved with said pathological condition. This target recognition segment comprises at least NKp46, NKp30, NKp44, or a biologically functional fragment thereof. The active segment of the complex may be selected from cytotoxic agents moieties and Ig fragments.

In one embodiment the composition of the present invention is intended for treating a malignant disease such as, for example, melanoma, carcinoma, sarcoma and lymphoma.

Alternatively, the compositions of the present invention are intended for treating viral infections caused by any one of Influenza virus, human immunodeficiency virus, Epstein-Barr virus, cytomegalovirus, Vaccinia virus, ECMV, MVM and Herpes virus.

The NKp46, NKp44 or their fragments, comprised in the complexes and compositions of the invention are capable of binding to a ligand expressed on the surface of the target cell.

When the target cell is a virus-infected cell, the binding is mediated by sialic acid. The complex of the invention may bind to a free virus, this binding also being mediated by sialic acid.

Preferably, the target recognition segment in the complexes and compositions of the invention comprises a NKp46 fragment comprising at least one of domains 1 and 2 of the NKp46 molecule, preferably comprising both domains 1 and 2. In another preferred embodiment the fragment comprising only domain 2 of NKp46. Alternatively, the target recognition segment in the complex and compositions of the invention comprises a NKp44 fragment comprising at least one of domains 1 and 2 of the NKp44 molecule, preferably comprising both domains 1 and 2. In another preferred embodiment the fragment comprising only domain 2 of NKp44.

The active segment of the complex or compositions of the invention may be an Ig fragment, preferably the Fc portion of an Ig molecule. Alternatively, the active fragment may be a cytotoxic moiety, such as a cytotoxin or an anticellular agent capable of killing and/or suppressing the growth and/or cell division of the target cell.

The cytotoxin or anticellular agent may be a synthetic agent or a plant-derived, fungal, or bacteria-derived toxin.

More particularly, the toxin may be selected from the group consisting of A chain toxin, ribosome inactivating protein, α-sarcin, aspergillin, restrictocin, ribonuclease, diphtheria toxin, Pseudomonas exotoxin, an endotoxin or the lipid A moiety of an endotoxin.

An alternative aspect of the present invention relates to a diagnostic composition for detecting the presence of diseased or defective cells in a sample. This diagnostic composition comprises a complex comprising a target recognition segment that is capable of specifically recognizing and binding to a diseased target cell involved with the pathological condition, and a detectable moiety. The recognition segment comprises NKp46, NKp44, NKp30 or a biologically functional fragment thereof. The detectable imaging moiety may be a paramagnetic, radioactive or fluorogenic agent.

Another aspect of the present invention relates to a method for treating a pathological condition in a subject. This method comprises the step of administering to the subject a pharmaceutically effective amount of a therapeutic agent comprising a complex having a first, target recognition segment, capable of specifically recognizing and binding to a diseased target cell involved with the pathological condition, and a second, therapeutically active segment. The target recognition segment comprises at least one of NKp46, NKp30, NKp44 or a biologically functional fragment thereof, and the therapeutically active segment may be a cytotoxic moiety or an Ig fragment. The pathological condition to be treated may be a viral infection caused by Influenza virus, human immunodeficiency virus, Epstein-Barr virus, cytomegalovirus, Vaccinia virus and Herpes virus, or a malignant disease such as melanoma, carcinoma, lymphoma and sarcoma.

In a specifically preferred embodiment, the method of the invention employs complexes in which NKp46 or NKp44 comprised in the target recognition segment are capable of binding to a ligand expressed on the surface of said target cell. When the target cell is virus infected cell this binding is mediated by sialic acid. More specifically, the NKp46 fragment comprises at least one of domains 1 and 2 of the NKp46 molecule, more preferably both domains 1 and 2. In another prefrred embodiment this fragment comprises only domain 2 of the NKp46 molecule. Alternatively, the NKp44 fragment comprises at least one of domains 1 and 2 of the NKp44 molecule, more preferably both domains 1 and 2. In another prefrred embodiment this fragment comprises only domain 2 of the NKp44 molecule.

In one alternative embodiment, the method of invention employs a complex containing as the active segment an Ig fragment. This Ig fragment is particularly the Fc portion of an Ig molecule.

In another alternative embodiment, the method of the invention comprises as an active segment a cytotoxic moiety. This cytotoxic moiety may be selected from cytotoxins or anticellular agents capable of killing and/or suppressing the growth and/or cell division of the target cell. More specifically, the cytotoxin or anticellular agent may a synthetic toxin or a plant-, fungus-, or bacteria-derived toxin.

This toxin may be selected from the group consisting of A chain toxin, ribosome inactivating protein, α-sarcin, aspergillin, restrictocin, ribonuclease, diphtheria toxin, Pseudomonas exotoxin, an endotoxin or the lipid A moiety of an endotoxin.

An alternative aspect of the present invention relates to a method for the diagnosis and imaging of pathologies, specifically tumors. This method comprises the steps of introducing an imaging agent into the blood stream of a subject, and detecting and quantitating the binding of the imaging agent to a NKp46 or NKp44 ligand expressed on malignant cells. The imaging agent comprises a complex having a first, target recognition segment, capable of specifically recognizing and binding to malignant cells. The recognition segment comprises at least one of NKp46, NKp30, NKp44 or a biologically functional fragment thereof. The complex also comprises a second, active segment, which is an imaging moiety, which may be a paramagnetic, radioactive or fluorogenic agent.

### BRIEF DESCRIPTION OF THE FIGURES

FIG. 1—Upregulation of NKp46 ligand expression post various virus infection

721.221 cells (10⁶/ml) were incubated overnight with 100 μl/ml of Sendai virus-containing supernatant. A9 cells (half-confluent culture flask passaged 24 hr before) were incubated for 3 hr with either MVM (1.5×10⁷ units/ml), EMCV (250 μl/ml of EMCV-containing supernatant), Adenovirus (5×10⁶ units/ml) or Vaccinia (4 ml Vaccinia-containing supernatant). Following incubation of A9 cells with the different viruses, virus-containing media were removed and culture media was added. 18 to 24 hr after infection cells (infected or uninfected) were harvested, washed and stained either with the NKp46-Ig fusion protein (bold line) or with control CD99-Ig (plain line), followed by PE-conjugated anti-human Fc. Numbers inside Panels represent the MFI of the NKp46-Ig staining. Results are from a representative experiment out of two performed. Abbreviations: Cou is for counts.

FIGS. 2A-C—NKp46 Ig mediated enhanced lysis of 293T cells transfected with the Sendai HN cDNA

FIG. 2A: NKp46-Ig binding to 293T cells transfected with Sendai HN cDNA. 293T cells were either transiently transfected with a control (cont) PCDNA3 plasmid (293T/MOCK) or with a cDNA coding for HN of the Sendai Virus (293T/pca-svhn). 48 hr later cells were stained either with TC-1D6 mAb or with KIR-1, NKAT-8, CD16 and NKp46 Ig-fusion proteins. MFI indicates Median Fluorescence Intensity. Controls were the same cells stained either with FITC-conjugated anti-mouse (anti=α) antibodies (No mAb), or with PE-conjugated anti-human Fc antibodies (no Ig-fusion protein). Results are of a representative experiment out of three performed.

FIGS. 2B and 2C: Enhanced lysis of 293T cells transfected with the Sendai HN cDNA is blocked by anti-NKp46 and anti-HN mAb. 48 h after transfection, 293T, 293T/MOCK and 293T/pca-svhn cells were labeled with ³⁵S-Met and washed. Labeled cells were then incubated, at the effector to target (E:T) ratios indicated, with NK GAL pre-incubated with either control serum or with anti-NKp46 serum (B). Alternatively, labeled cells were incubated with the various mAbs for 1 hr on ice, washed, and then incubated with NK GAL (C). In all experiments NK cells were pre-incubated with 50% of human serum (ser) for 1 h on ice and then washed to block Fc receptors. Results are representative experiment of three performed. Abbreviations: Prot (protein), specific lysis (sp ly), ratio (ra), MO (mock).

FIGS. 3A-C—Lysis of IV-infected 1106mel cells by NK GAL and derived clones

FIG. 3A: NK cells were incubated either with no antibody, with the control (cont) anti-CD99 mAb (12E7), with anti-CD16 mAb (anti=α) (3G8), with control serum (cont ser), or with anti-NKp46 serum, for 1 h on ice. NK cells were then washed and then incubated either with 1106mel cells or with IV-infected 1106mel cells at the E:T ratios (=ra) indicated.

FIG. 3B: IV-infected 1106mel cells were incubated with various mAb for 1 hr on ice and then incubated with NK GAL at the indicated E:T ratios. Results are representative experiment of three performed.

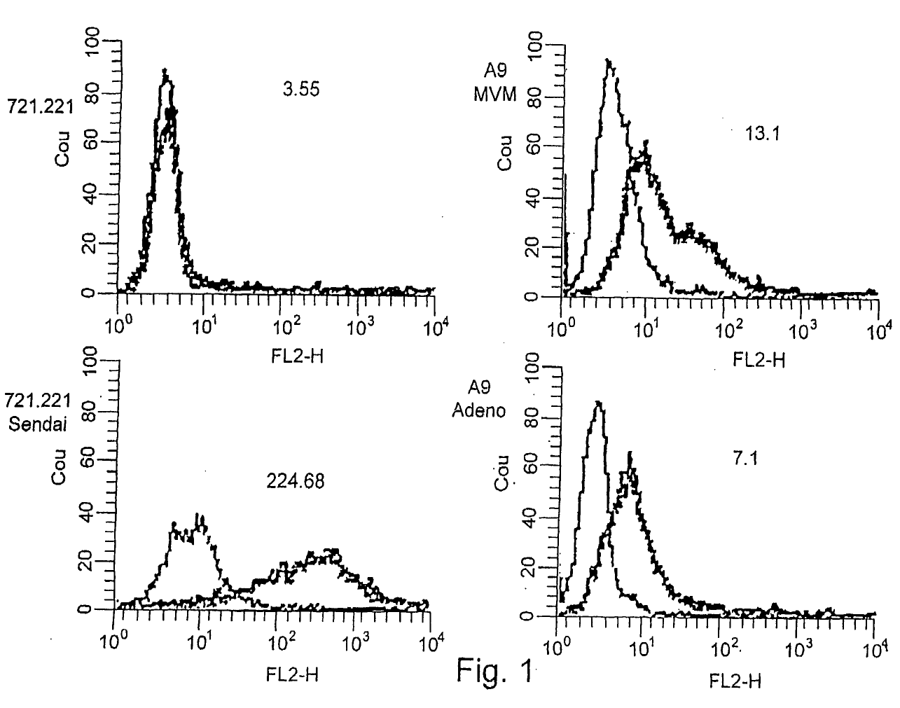

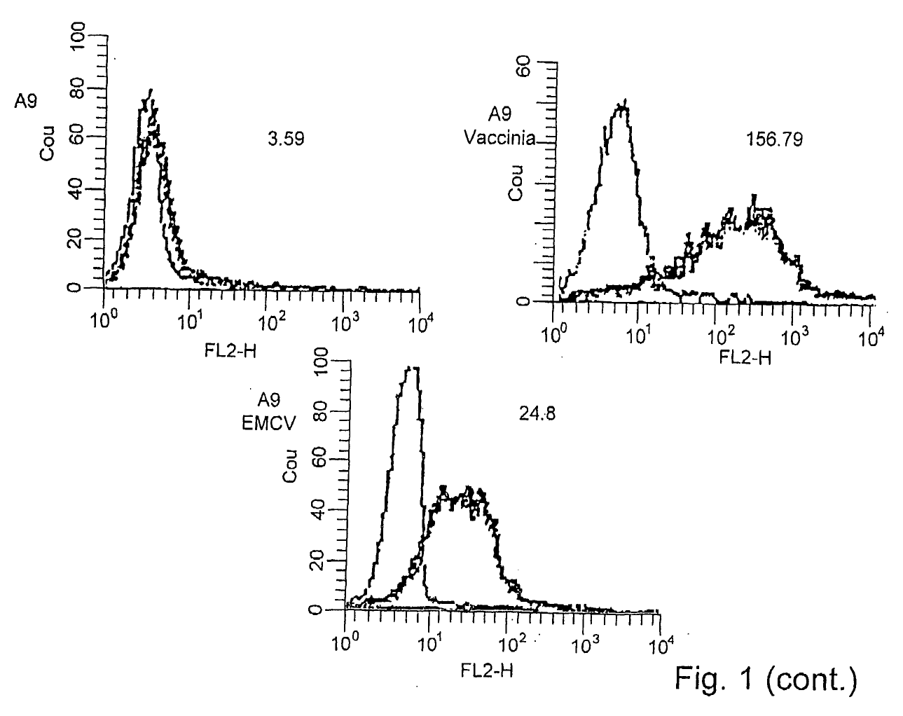

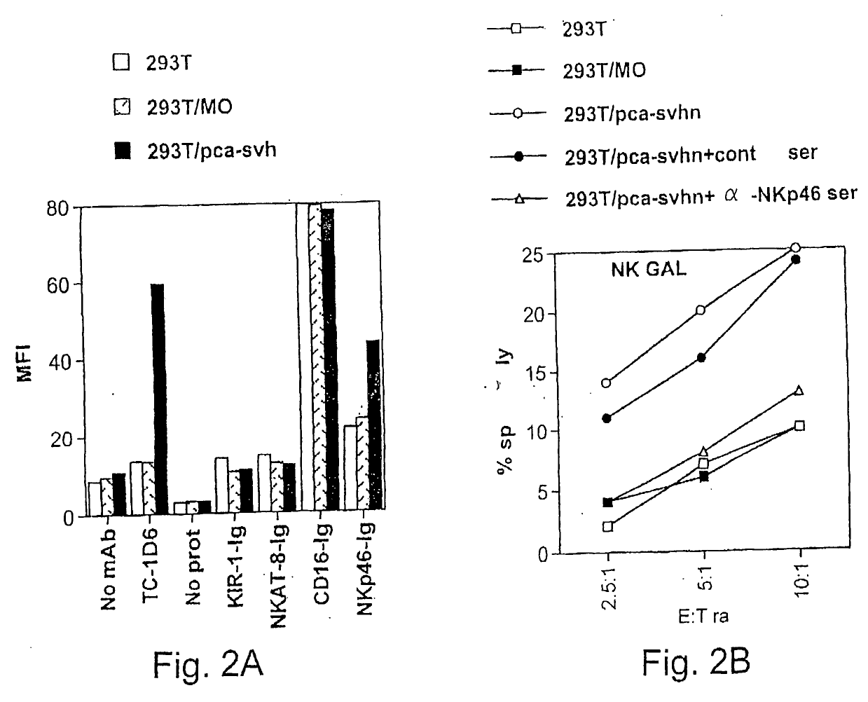

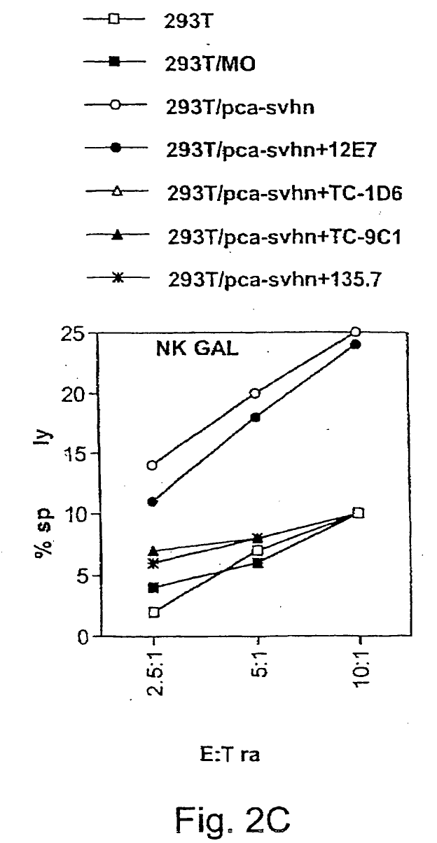

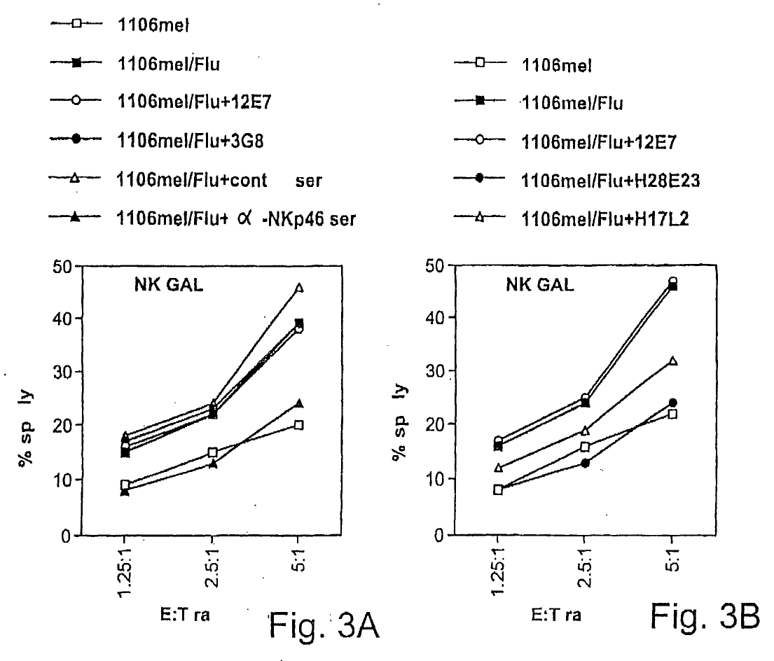

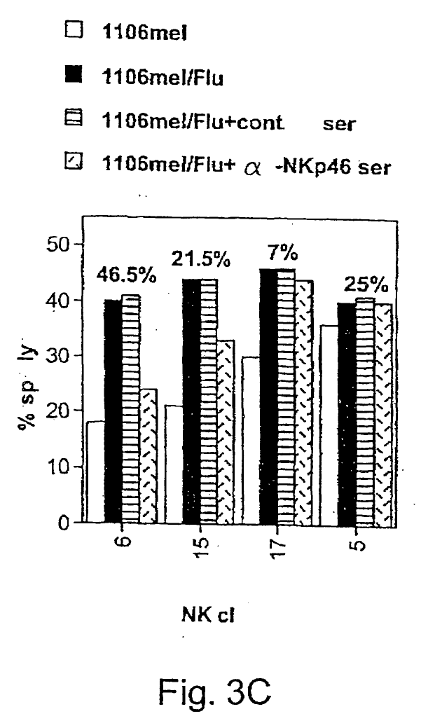

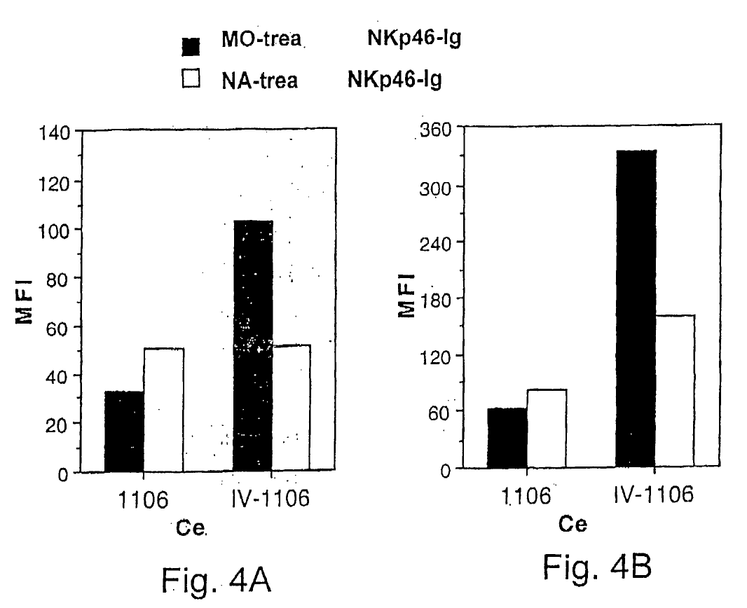

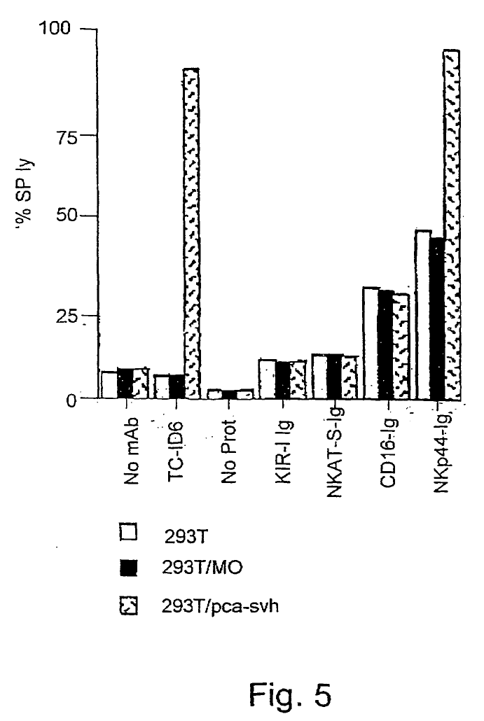

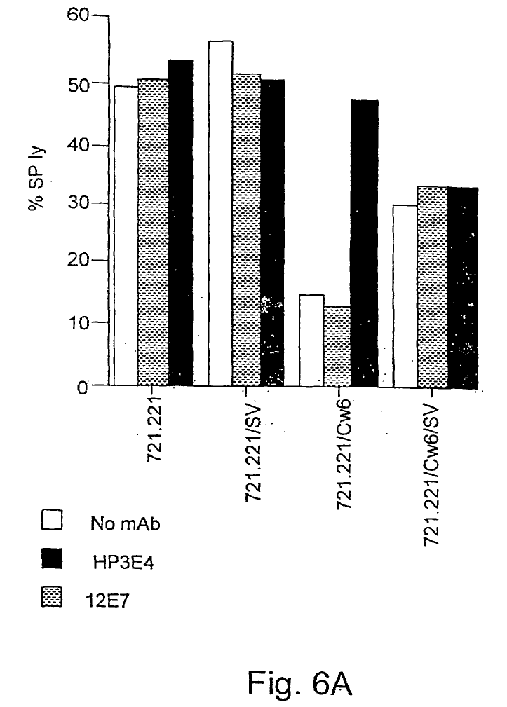

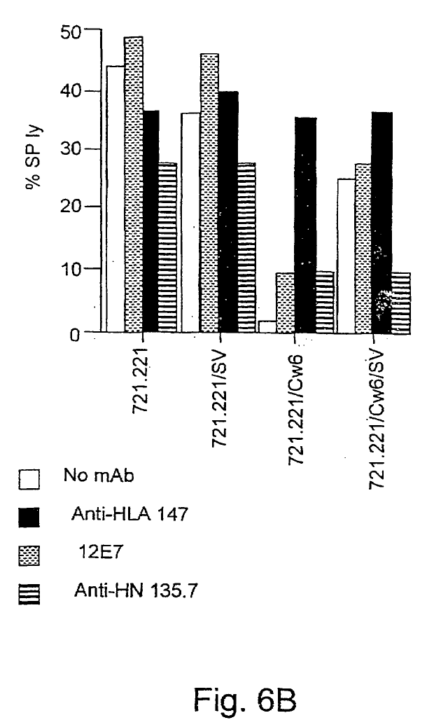

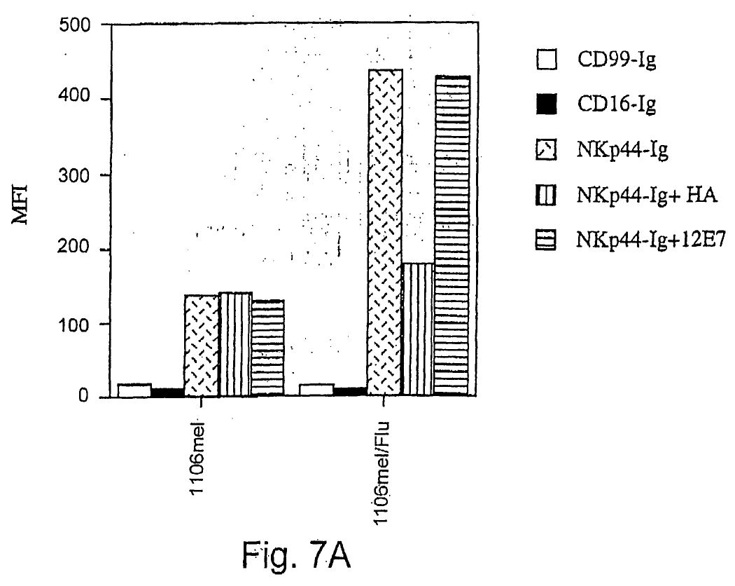

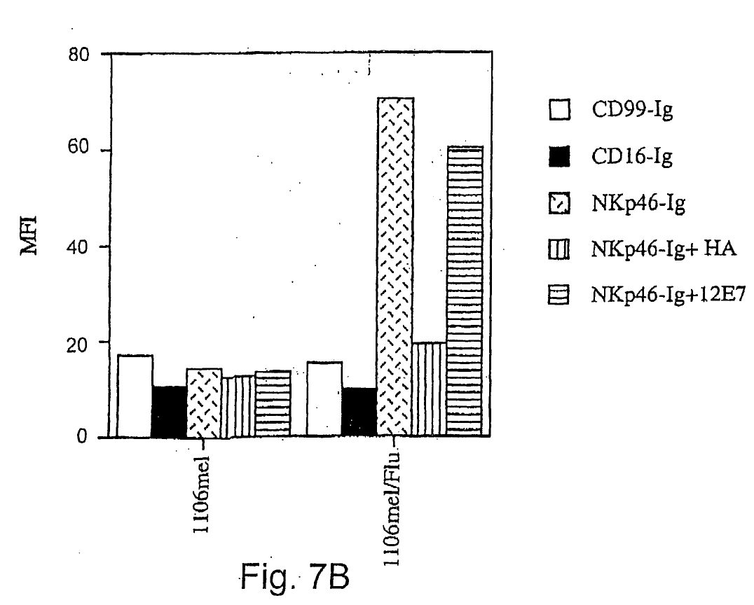

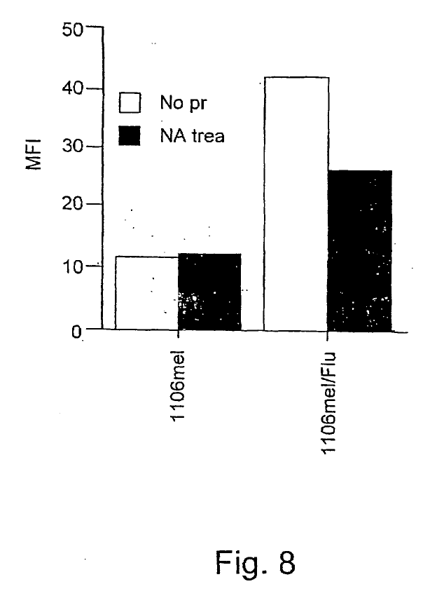

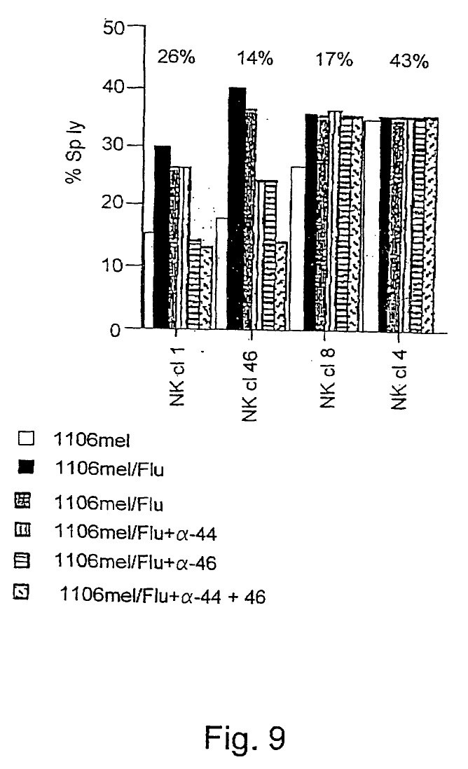

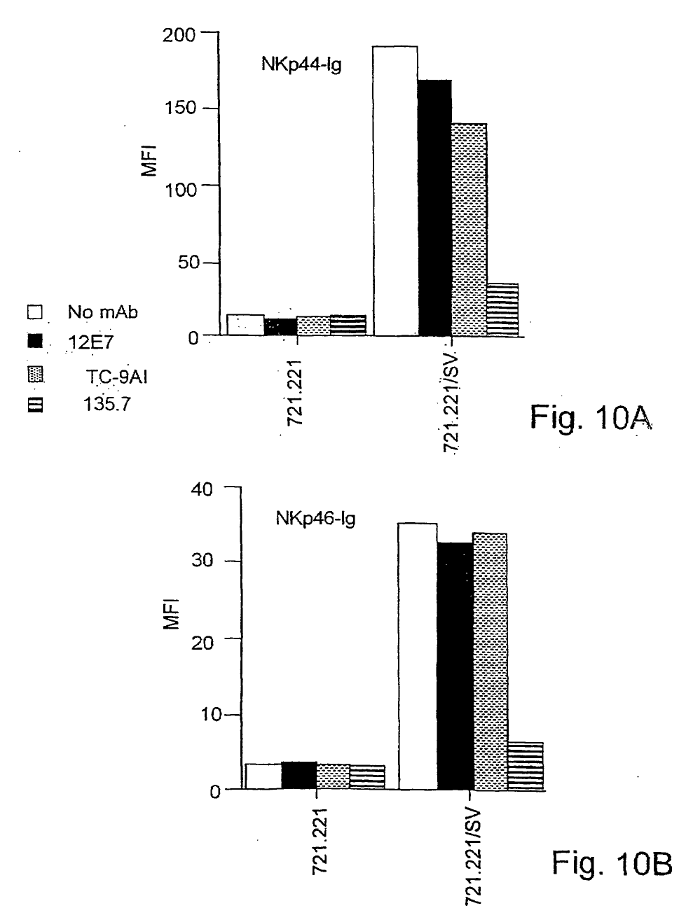

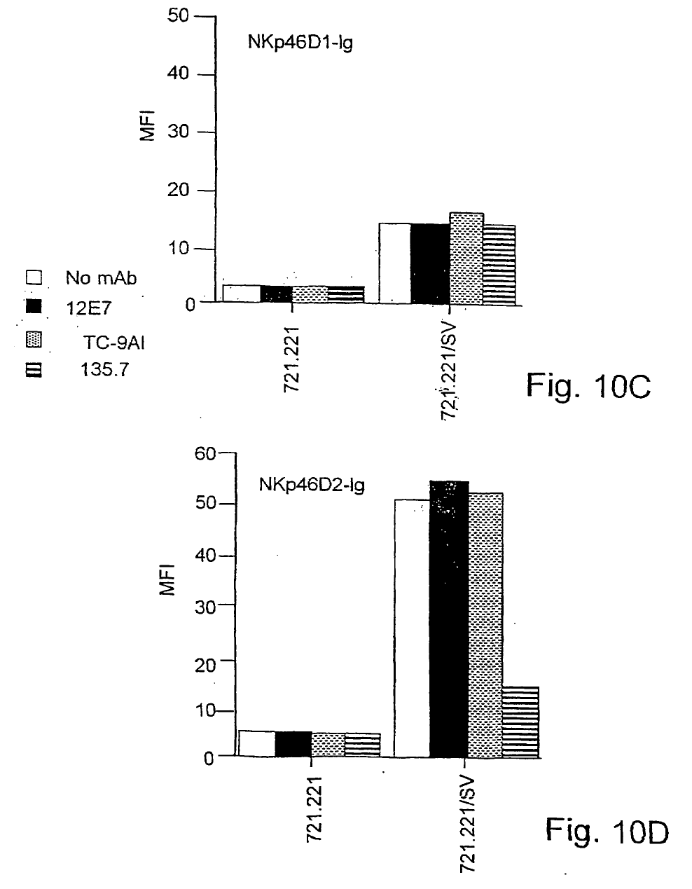

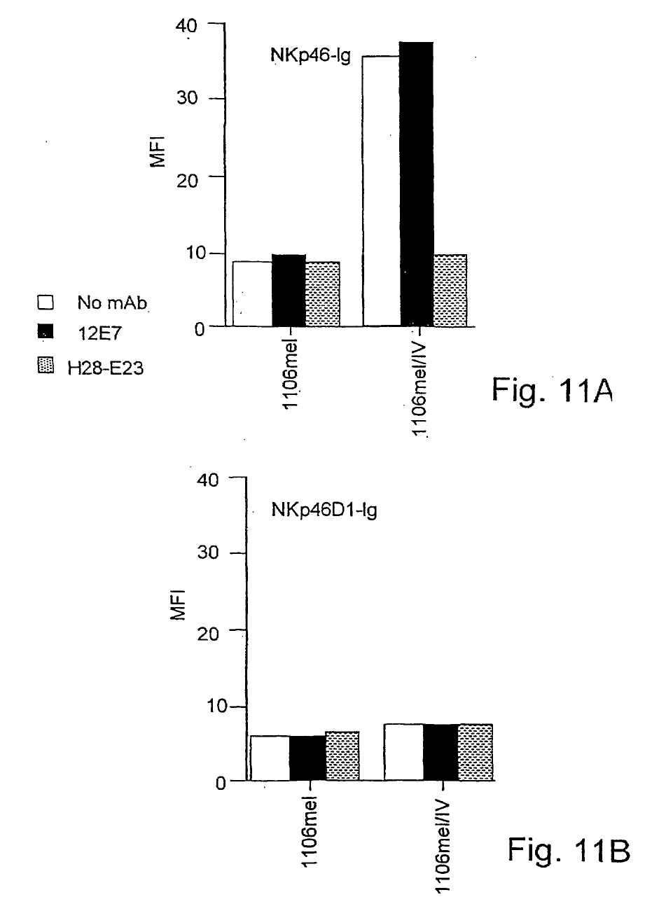

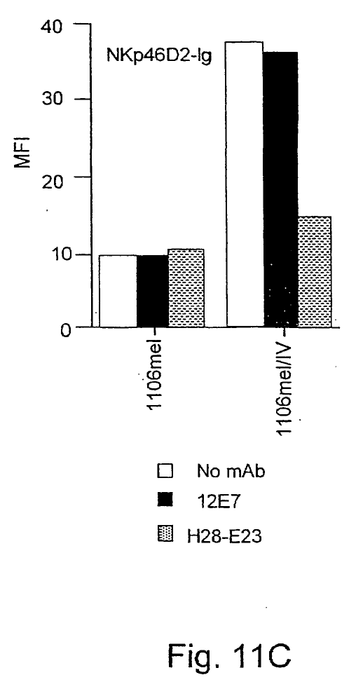

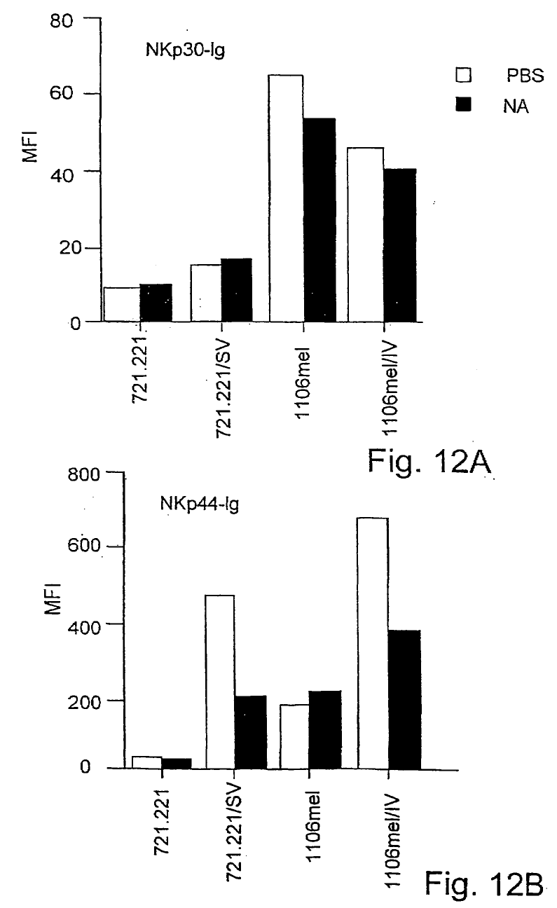

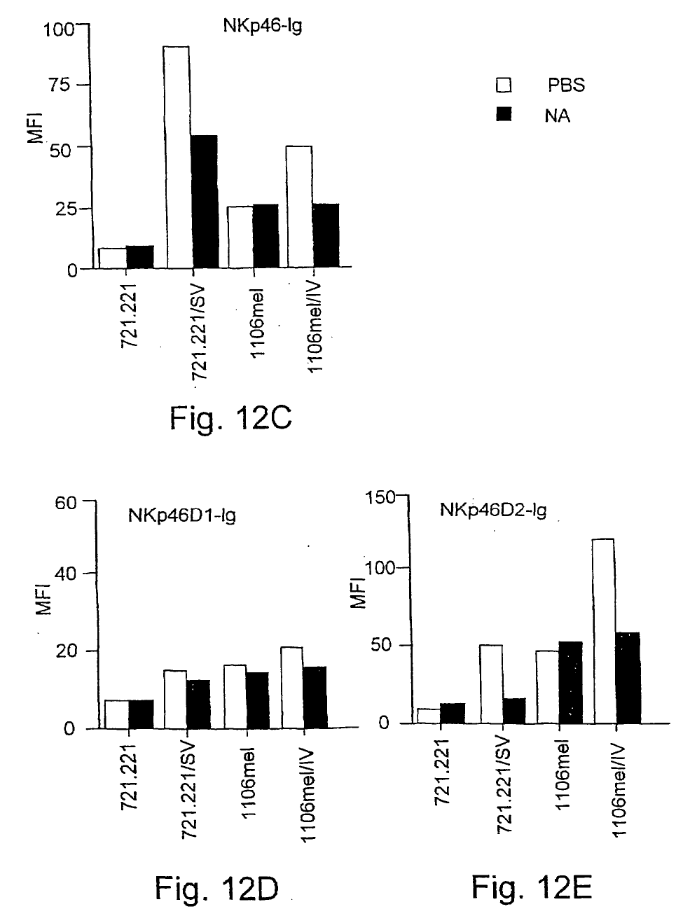

FIG. 3C: 28 NK clones were derived from the NK GAL by limiting dilution. Blocking experiments with serum containing polyclonal antibodies were performed as described in legend to FIG. 1. The E:T ratio was 5:1. The percentages indicated in the Figure are the % of clones that behaved similarly to the one NK clone presented. Results are representative experiment of two performed. In all experiments NK cells were pre-incubated with 50% of human serum for 1 hr on ice and then washed to block Fc receptors. Abbreviations: sp ly is for specific lysis, cont (control), ser (serum) and cl is for clones.

FIG. 4—Effect of NA treatment on the binding of NKp46-Ig to IV-infected and non-infected 1106 cells

NKp46-Ig was incubated with 0.01 U of insoluble neuraminidase attached to beaded agarose (N-5254, SIGMA, St. Louis, Mo.) or with PBS (mock treated—MO-trea) for 1 h at 17° C. on a roller. IV-infected, or uninfected 1106 cells (Ce) were washed, and stained either with NA-treated (trea) or mock-treated NKp46-Ig, followed by PE-conjugated anti-human Fc. MFI indicates Median Fluorescence Intensity. A and B panels are two representative experiments of eight performed. The activity of NA was confirmed by SDS-PAGE analysis of NA-treated fetuin, a highly sialylated protein.

FIG. 5—NKp44-Ig binding to 293T cells transfected with Sendai HN cDNA

293T cells were either transiently transfected with a control PCDNA3 plasmid (293T/MOCK) or with a cDNA encoding for HN of the SV (293T/pca-svh) using the Fugene transfection reagent (Boehringer Mannheim). 48 hr later cells were stained either with TC-1D6 mAb or with KIR-1, NKAT-8, CD16 and NKp46 Ig-fusion proteins. MFI indicates Median Fluorescence Intensity. Controls were the same cells stained either with FITC-conjugated anti-mouse antibodies (No mAb), or with PE-conjugated anti-human Fc antibodies (no Ig-fusion protein). Results are of a representative experiment out of two performed. Abbreviations: sp ly is for specific lysis, MO (MOCK), prot (protein).

FIGS. 6A-B—SV Infection of 721.221 expressing class I MHC proteins resulted in abrogation of the inhibition

NK clones were derived from various donors using the autoMACS instrument (Miltenyi Biotec Inc). Clones were stained for the presence of NKp44 and NKp46 proteins using the anti-NKp44 and NKp46 serum and for the presence of NK inhibitory receptors using the HP3E4 mAb. The E: T ratio of the NK clone presented was around 3:1. Results are representative experiment of two performed.

FIG. 6A: shows the NK clone was first incubated with the indicated mAb for 1 hr and then incubated with the labeled target cells.

FIG. 6B: shows labeled target cells that were first incubated with the indicated mAb for 1 hr on ice and then incubated with NK cells.

Abbreviations: sp ly is for specific lysis.

FIG. 7—Effect of HA blocking on the binding of NKp46-Ig and NKp44-Ig to IV-infected and non-infected 1106mel cells

NKp44-Ig (upper panel) or NKp46-Ig (lower panel) were incubated with or without 40 μg purified HA protein. Mixtures were next incubated with IV-infected or non-infected 1106mel cells and stained with PE-conjugated goat anti-human Fc. MFI indicates Median Fluorescence Intensity.

FIG. 8—Effect of NA treatment on the binding of NKp44-Ig to IV-infected and non-infected 1106mel cells

MFI indicates Median Fluorescence Intensity. NKp44-Ig was incubated with 0.01U of insoluble neuraminidase attached to beaded agarose (N-5254, SIGMA, St. Louis, Mo.) or with PBS (as control) for 1 h at 17° C. on a roller. IV-infected, or non-infected 1106mel cells were washed, and stained either with NA-treated or mock-treated NKp44-Ig, followed by PE-conjugated anti-human Fc. Figure shows one representative experiment of two performed. The activity of NA was confirmed by SDS-PAGE analysis of NA-treated fetuin, a highly sialylated protein. Abbreviations: N pr (no protein) and NA trea (NA treatment).

FIG. 9—Lysis of IV-infected 1106mel cells by NK clones 64 NK clones were derived from the NK line MB by limiting dilution. Blocking experiments with serum containing polyclonal antibodies were performed as described in experimental procedures. The E:T ratio was 2:1. The percentages indicated in the Figure are the % of clones that behaved similarly to the one NK clone presented. 1106mel/Flu+C indicates incubation with control serum. Results are representative experiment of two performed. In all experiments NK cells were pre-incubated with 50% of human serum for 1 hr on ice and then washed to block Fe receptors. Abbreviations: sp ly (specific lysis), cl (clon) and α (anti).

FIGS. 10A-D—Domain 2 is responsible for the interaction with the HN viral protein of SV

721.221 cells were infected with 100 μl of SV supernatant. After overnight incubation, infected cells were washed and incubated on ice with the various mAbs, for 1 h. Next, cells were washed and assayed for staining with 10 μg of the appropriate Ig-fusion protein as previously described [Mandelboim, O., et al., (1999) ibid.], MFI indicates Median Fluorescence Intensity.

FIGS. 11A-C—Domain 2 is responsible for the interaction with the HA viral protein of IV

1106mel cells were infected with 1000 u/ml of IV. After overnight incubation, infected cells were washed and incubated on ice with the various mAbs, for 1 h. Next, cells were washed and assayed for staining with 10 μg of the appropriate Ig-fusion protein as previously described [Mandelboim, O., et al., (1999) ibid.]. MFI indicates Median Fluorescence Intensity.

FIGS. 12A-E—Effect of NA treatment on the binding of NKp46d2-Ig to infected and non-infected cells

The different indicated fusion proteins were incubated with 0.01 U of insoluble neuraminidase (NA) attached to beaded agarose (N-5254, SIGMA, St. Louis, Mo.) or with PBS (mock treated) for 1 h at 17° C. on a roller. IV-infected, or uninfected 1106 cells, as well as SV-infected, or non-infected 721.221 cells were washed, and stained either with NA-treated (trea) or PBS-treated fusion proteins, followed by PE-conjugated anti-human Fc. MFI indicates Median Fluorescence Intensity. The activity of NA was confirmed by SDS-PAGE analysis of NA-treated fetuin, a highly sialylated protein.

FIG. 12A: shows incubation with the NKp30-Ig fusion protein.

FIG. 12B: shows incubation with the NKp44-Ig fusion protein.

FIG. 12C: shows incubation with the NKp46-Ig fusion protein.

FIG. 12D: shows incubation with the NKp46D1-Ig fusion protein.

FIG. 12E: shows incubation with the NKp46D2-Ig fusion protein.

### DETAILED DESCRIPTION OF THE INVENTION

A number of methods of the art of molecular biology are not detailed herein, as they are well known to the person of skill in the art. Such methods include site-directed mutagenesis, PCR cloning, expression of cDNAs, analysis of recombinant proteins or peptides, transformation of bacterial and yeast cells, transfection of mammalian cells, and the like. Textbooks describing such methods are e.g., Sambrook et al., Molecular Cloning A Laboratory Manual, Cold Spring Harbor Laboratory; ISBN: 0879693096, 1989, Current Protocols in Molecular Biology, by F. M. Ausubel, ISBN: 047150338X, John Wiley & Sons, Inc. 1988, and Short Protocols in Molecular Biology, by F. M. Ausubel et al. (eds.) 3rd ed. John Wiley & Sons; ISBN: 0471137812, 1995. These publications are incorporated herein in their entirety by reference. Furthermore, a number of immunological techniques are not in each instance described herein in detail, as they are well known to the person of skill in the art. See e.g., Current Protocols in Immunology, Coligan et al. (eds), John Wiley & Sons. Inc., New York, N.Y.

The present invention provides a novel approach to the treatment and/or diagnosis (imaging) of different pathologies such as tumors and pathogenic viral infections, based on recognition of different ligands expressed on tumor or viral infected cells, by the NK cells activating receptors NKp46, NKp44 and NKp30.

In diagnosis, the method of the invention will have the ability to provide an image of the tumor, for example through magnetic resonance imaging, X-ray imaging, computerized emission tomography and the like, by means of a complex of the invention comprising a detectable imaging moiety.

In therapy, complexes of the invention are designed to have a cytotoxic or otherwise anticellular effect against desired target cells, by suppressing the growth or cell division of such cells.

Thus, in a first aspect, the present invention relates to a targeting complex that specifically recognizes a ligand molecule expressed on the surface of target cells and is capable of targeting an active substance to the target cell. This complex comprises:

a. a target recognition segment comprising at least one of the NKp46, NKp30 and NKp44 proteins or a functional fragment thereof; and

b. an active segment comprising the active substance which may be a cytotoxic moiety, an imaging moiety or an Ig fragment

In a specifically preferred embodiment the target recognition segment is a NKp46 fragment which comprises at least one of domains 1 and 2 of the NKp46 molecule. Preferably, the target recognition segment comprises both domains 1 and 2 and has the amino acid sequence substantially as denoted by SEQ ID NO: 4 or the amino acid sequence of its isoform, substantially as denoted by SEQ ID NO: 13. In another preferred embodiment, the target recognition segment may comprise only domain 2 of the NKp46 molecule, as denoted by SEQ ID NO: 22 and 23.

Alternatively, the target recognition segment may be a NKp44 fragment which comprises at least one of domains 1 and 2 of the NKp44 molecule. Preferably the target recognition segment comprises both domains 1 and 2 and has the amino acid sequence substantially as denoted by SEQ ID NO: 9. In another preferred embodiment, the target recognition segment may comprise only domain 2 of the NKp44 molecule, as denoted by SEQ ID NO: 24. The target recognition segment may alternatively comprise the NK activating molecule NKp30, as denoted by SEQ ID NO: 17.

It is to be appreciated that the recognition segment of the invention may comprise more than one unit of domain 1 and 2 or alternatively only domain 2 of any of NKp46, NKp30 and NKp44. Creation of such multiunit segment may increase the avidity of the recognition segment to the target molecule.

By “functional fragments” is meant “fragments”, “variants”, “analogs” or “derivatives” of the molecule. A “fragment” of a molecule, such as any of the nucleic acid or the amino acid sequence of the present invention is meant to refer to any nucleotide or amino acid subset of the molecule. A “variant” of such molecule is meant to refer to a naturally occurring molecule substantially similar to either the entire molecule or a fragment thereof. An “analog” of a molecule is a homologous molecule from the same species or from different species. The amino acid sequence of an analog or derivative may differ from the specific molecule, e.g. the NKp46, NKp30 or NKp44 molecule, used in the present invention when at least one residue is deleted, inserted or substituted.

By “functional” is meant having same biological function, for example, having identical ability to recognize and/or bind the ligand.

Another specifically preferred embodiment, relates to the complex the invention being a fusion protein comprising as the active segment an Ig fragment. This Ig fragment is preferably the Fc portion of an Ig molecule, and is encoded by the amino acid sequence of SEQ ID NO: 5.

The complement system is one of the major effector mechanisms of humoral immunity. It is activated principally by the binding of the first classical pathway component, C1, to the Fc portion of antigen-complexed antibody molecules. Therefore, the fusion proteins comprising NKp44, NKp30 or NKp46 and the Fc portion of an Ig molecule, can serve as target for the complement system in vivo.

Alternatively, the complex of the present invention is a conjugate comprising as an active segment a cytotoxic moiety. This cytotoxic moiety may be a cytotoxin or any anticellular agent, which is capable of killing and/or suppressing the growth or cell division of said target cell.

In general, for therapeutic purposes, the invention involves the use of any pharmacological agent that can be conjugated to the targeting segment of the complex of the present invention and delivered in active form to the target cells. Exemplary anticellular agents include chemotherapeutic agents, radioisotopes as well as cytotoxins. Chemotherapeutic agents include, but are not limited to, hormones such as steroids, antimetabolites such as cytosine arabinoside, fluorouracil, methotrexate or aminopterin; an anthracycline; mitomycin C; a Vinca alkaloid; demecolcine; etoposide; mithramycin; or an antitumor alkylating agent such as chlorambucil or melphalan.

Other embodiments may include agents such as bacterial endotoxins or the lipid A moiety of such bacterial endotoxin. In any event it is proposed that agents such as these may be successfully conjugated to the targeting segment (preferably any one of NKp46, NKp44 and NKp30 domains 1 and 2 or at least one of domains 1 and 2, preferably domain 2) of the complex of the present invention, in a manner that will allow their targeting, internalization, release or presentation to the target cells as required, using known conjugation technologies [for example, Ghose, et al., Critical Reviews in Therapeutic Drug Carrier Systems, 3:256-359 (1987)].

In certain preferred embodiments, agents for therapeutic application will include generally a synthetic toxin or a plant-, fungus-, or bacteria-derived toxin, such as an A toxin, a ribosome inactivating protein, α-sarcin, aspergillin, restirictocin, a ribonuclease, diphtheria toxin, Pseudomonas exotoxin, an endotoxin or the lipid A moiety of an endotoin, to mention just a few examples. The most preferred toxin moiety for use in connection with the invention is diphtheria toxin.

It is contemplated that most therapeutic applications of the present invention will involve the targeting of a toxin moiety to the tumor or to the pathogenic virus-infected cells. This is due to the much greater ability of most toxins to deliver a cell killing effect as compared to other potential agents. Nevertheless, under some circumstances, such as when the target ligand of the NKp46, NKp44 or NKp30 does not internalize via a route consistent with efficient intoxication by targeted toxin-complexes, where toxins one may be substituted by chemotherapeutic agents such as antitumor drugs, other cytokines, antimetabolites, alkylating agents, hormones, and the like.

As used in the present application, the terms “cytotoxic” and “cytolytic” when used to describe the activity of the complex of the present invention (particularly when a cytotoxic moiety is selected as the active segment) are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell.

In yet another alternative embodiment, the complex of the present invention may be a conjugate comprising as the active segment an imaging moiety. Moreover, in the case of radioactive isotopes for therapeutic and/or diagnostic application, one might mention iodine¹³¹, iodine¹²³, technecium^{99m}, indium¹¹¹, rhenium¹⁸⁸, rhenium¹⁸⁶, galium⁶⁷, copper⁶⁷, yttrium⁹⁰, iodine¹²⁵ or astatine²¹¹. Where the aim is to provide an image of the tumor for diagnosis and monitoring purposes, one will desire to use an agent that is detectable upon imaging, such as a paramagnetic, radioactive or fluorogenic agent. Many agents are known in the art to be useful for imaging purpose. Paramagnetic ions may be, for example, ions such as chromium, manganese, iron, cobalt, nickel, copper, neodymium, samarium, holmium or erbium. Ions useful in other contexts, such as x-ray imaging, include but are not limited to lanthanum, gold, lead and bismuth.

The imaging complex of the invention may be conjugated to a detectable moiety such fluorescent compound. When the fluorescently-labeled complex is exposed to light of the proper wavelength, its presence can be detected by fluorescence. Amongst the most commonly used fluorescent labeling compounds are fluorescein, isothiocyanate, rhodamine, phycoerythrine, phycocyanin, allophycocyanin, o-phthaldehyde and fluorescamine.

The complex can also be detectably labeled using fluorescence emitting metals such as ¹⁵²E, or others of the lanthanide series. These metals can be attached to the targeting segment using such metal chelating groups as diethylenetriamine pentaacetic acid (ETPA).

The complex can also be detectably labeled by being coupled to a chemiluminescent compound. The presence of the chemiluminescent-tagged targeting segment is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used as a label in the complex of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

It is to be appreciated that the targeting segment of the complex of the invention may be conjugated to the active segment (cytotoxic or imaging moieties), either directly or indirectly by conjugating or coupling these segments to any one of lipid backbone or carbohydrate backbone.

In a specific embodiment, the complex of the present invention may be targeted to a diseased cell.

As used to describe the present invention, “target cells” are the cells that are killed by the cytotoxic activity of the complex of the invention (wherein the active segment comprises a therapeutic, e.g., cytotoxic moiety or Ig), or cells detected by the complex of the invention (where the active segment comprises a detectable imaging moiety). The target cells express the ligand for any one of NKp46, NKp44 and NKp30 molecules and include, in particular, cells that are malignant or otherwise derived from solid as well as non-solid tumors.

As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the complex of the present invention as well as the composition and the methods of the present invention may be used in the treatment of non-solid and solid tumors, and for monitoring and imaging of solid tumors (wherein the selected active segment is an imaging moiety).

Alternatively, the complex of the present invention may be directed to cells that are infected by pathogenic viruses such as HIV, EBV, CMV, Vaccinia, MVM, ECMV, Herpes or Influenza virus.

More specifically, the pathogenic virus may be any one of variety of viruses including but not limited to, Influenza virus, human immunodeficiency virus, Epstein-Barr virus, cytomegalovirus, Vaccinia virus, MVM, ECMV and Herpes virus.

As used to describe the present invention, a “pathogenic virus” is a virus causing disease in a host. The pathogenic virus infects cells of the host animal and the consequence of such infection is deterioration in the health of the host. Pathogenic viruses envisioned by the present invention include, but are not limited to, HIV, EBV, CMV, Vaccinia, Herpes, MVM, ECMV and Influenza.

In a specifically preferred embodiment, where said target cell is a pathogenic virus-infected cell, the complex of the invention has a target recognition segment that is capable of binding to a ligand expressed on the surface of said viral infected target cell, this binding is mediated by sialic acid.

Alternatively, the complex of the invention may also bind to a free virus. This binding as well is mediated by sialic acid.

As shown in Examples 6 and 9, the interaction of the NKp46 and NKp44 molecules, respectively, with their ligands (HA or HN) was mediated by sialic acid. Moreover, it was shown that sialic acid is necessary but not sufficient for the interaction.

A number of mammalian sialic acid receptors have been defined [Varki, A., et al, FASEB J. 11:248-55 (1997)], raising the question of why these are not sufficient for binding NKp46 or NKp44. While cellular sialic acid receptors may be expressed on the target cells that were utilized, they may not be expressed in sufficient quantities to function like viral HA, which is abundantly expressed. It is also possible that the cellular lectins are sequestered or otherwise inactivated until the appropriate circumstances arise for their use.

Examples 6 and 9 describe the analysis of the binding of HA to NKp46 and NKp44. The dissociation constant of HA for sialic acids is in the mM range, too low for a monomeric interaction to account for either the stable binding of NKp46-Ig detected by flow cytometry (which requires dissociation constants less than 0.1 μM), or the potency of NKp46-Ig in blocking viral hemagglutination. This implies either a multimeric interaction of HA with NKp46 or that NKp46 interacts more intimately with HA after contact is initiated by the sialic acid binding. Even in the former case, NKp46 would need special properties to distinguish it from other cellular glycoproteins, since terminal sialic residues are ubiquitous on N-linked oligosaccharides. One possibility is that NKp46 (which is thought to have a single N-linked oligosaccharide) multimerizes in such a fashion as to enable multivalent interaction of sialic acid with a single HA complex, which as a trimer possesses three sialic acid binding sites. While recombinant NKp46-Ig and NKp44-Ig are expected to be bivalent molecules, the oligomeric state of cellular NKp46 or NKp44 is unknown.

In a second aspect, the present invention relates to an expression vector comprising a nucleic acid sequence coding for a NKp46-Ig fusion protein. This nucleic acid comprises:

a. a DNA sequence encoding a target recognition segment, which segment comprises NKp46 or functional fragments thereof. This NKp46 fragment preferably comprises at least one of domains 1 and 2 of the NKp46 molecule. Most preferably, the target recognition segment comprises both domains 1 and 2 of the NKp46 molecule and is encoded by the nucleic acid sequence substantially as denoted by SEQ ID NO: 1; or by the NKp46 isoform substantially as denoted by SEQ ID NO: 11, alternatively only domain 2 as denoted by any one of SEQ ID NO: 19 and NO: 20, and

b. a DNA sequence encoding an active segment which is the Fc portion of an Ig molecule, said DNA having the nucleic acid sequence substantially as denoted by SEQ ID NO: 2.

In an alternative embodiment the expression vector of the present invention comprises a nucleic acid sequence coding for a NKp44-Ig fusion protein. This nucleic acid sequence comprises:

a. a DNA sequence encoding a target recognition segment, which segment comprises NKp44 or functional fragments thereof. This NKp44 fragment preferably comprises at least one of domains 1 and 2 of the NKp44 molecule. Most preferably, the target segment comprises both domains 1 and 2 and is encoded by the nucleic acid sequence substantially as denoted by SEQ ID NO: 7, alternatively, only domain 2 as denoted by SEQ ID NO: 21; and

b. a DNA sequence encoding an active segment which is the Fc portion of an Ig molecule, said DNA having the nucleic acid sequence substantially as denoted by SEQ ID NO: 2.

An expression vector coding for the NKp30-Ig fusion protein is whithin the scope of the present invention. Such expression vector comprises the nucleic acid sequence of the NKp30 domain 1 and 2 and have the nucleic acid sequence as denoted by SEQ ID NO: 15.

As used herein, the term “nucleic acid” refers to polynucleotides such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded and double-stranded polynucleotides.

The expression vector of the invention may further comprise operably linked regulatory elements. The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Accordingly, the term control and regulatory elements includes promoters, terminators and other expression control elements. Such regulatory elements are described by Goeddel [Goeddel, Gene Expression Technology: Methods in Enzymology 185, Academic Press, San Diego, Calif. (1990)].

“Vectors”, as used herein, encompass plasmids, viruses, bacteriophage, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or an eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector containing cells. Plasmids are the most commonly used form of vector but other forms of vectors which serves an equivalent function and which are, or become, known in the art are suitable for use herein. See, e.g., Pouwels et al. Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

Also, a specific embodiment of the invention relates to a host cell transformed with the expression vectors of the invention. Suitable host cells include prokaryotes, lower eukaryotes, and higher eukaryotes. Prokaryotes include gram negative and gram positive organisms, e.g., E. coli and B. subtilis. Lower eukaryotes include yeast, S. cerevisiae and Pichia, and species of the genus Dictyosteliun. Higher eukaryotes include established tissue culture cell lines from animal cells, both of non-mammalian origin, e.g., insect cells and birds, and of mammalian origin, e.g., human and other primate, and of rodent origin.

A specifically preferred embodiment relates to a NKp46-Ig fusion protein. This fusion protein has the amino acid sequence substantially as denoted by SEQ ID NO: 6 or by its isoform as denoted by SEQ ID NO: 14, encoded by the nucleic acid sequence substantially as denoted by SEQ ID NO: 3 and SEQ ID NO: 12, respectively.

In another embodiment, the invention relates to the NKp46D2-Ig fusion protein as described in Example 11.

Another specifically preferred embodiment relates to a NKp44-Ig fusion protein. This fusion protein comprises the amino acid sequence substantially as denoted by SEQ ID NO: 10, that is encoded by the nucleic acid sequence substantially as denoted by SEQ ID NO: 8. In another embodiment, the invention relates to the NKp44D2-Ig fusion protein.

Alternatively, the fusion protein of the invention may comprise as the targeting segment the NKp30 molecule (denoted by SEQ ID NO: 18).

A heterologous fusion protein is a fusion protein made of segments, which are naturally not normally fused in the same manner. Thus, the fusion product of the NKp46, NKp30 or NKp44 (particularly domains 1 and 2, or at least one of domains 1 and 2) molecule with the Fc portion of an Ig molecule, is a continuous protein molecule having sequences fused by a typical peptide bond, typically made as a single translation product and exhibiting properties derived from each source peptide.

Another aspect of the present invention relates to an antibody that specifically recognizes and binds to the fusion protein NKp46-Ig of the invention.

In yet another embodiment of the present aspect the invention relates to an antibody that specifically recognizes and binds to the fusion protein NKp44-Ig of the invention.

Additionally, the invention relates to an antibody that specifically recognizes and binds to an epitope on a protein, wherein said protein is a ligand for the NK cell activating receptor NKp46. As described in Example 3, in order to identify the NKp46 putative ligands, mice were immunized with SV-infected cells, and the different antibodies were examined for their ability to block binding of the NKp46-Ig to SV infected cells. One such antibody that efficiently blocked the binding of the NKp46-Ig to SV infected cells is designated 135.7.

Therefore, in a specifically preferred embodiment the invention relates to the antibody designated 135.7. This antibody specifically recognizes and binds to a NKp46 ligand. As described in Example 3, this ligand is a protein having a molecular weight of approximately 70 Kd. This protein was found out to be the HN glycoprotein.

A preferred embodiment of the invention relates to the antibodies against the NKp46-Ig and the NKp44-Ig fusion proteins and against the NKp46 and the NKp44 ligands. These antibodies are selected from the group consisting of monoclonal and polyclonal antibodies, preferably monoclonal antibodies.

The generation of polyclonal antibodies against proteins is described in Chapter 2 of Current Protocols in Immunology, Wiley and Sons Inc.

Monoclonal antibodies may be prepared from B cells taken from the spleen or lymph nodes of immunized animals, in particular rats or mice, by fusion with immortalized B cells under conditions which favor the growth of hybrid cells. For fusion of murine B cells, the cell line Ag-8 is preferred.

The technique of generating monoclonal antibodies is described in many articles and textbooks, such as the above-noted Chapter 2 of Current Protocols in Immunology. Spleen or lymph node cells of these animals may be used in the same way as spleen or lymph node cells of protein-immunized animals, for the generation of monoclonal antibodies as described in Chapter 2 therein. The techniques used in generating monoclonal antibodies are further described by Kohler and Milstein, Nature 256:495-497, (1975), and in U.S. Pat. No. 4,376,110.

The term “antibody” is meant to include both intact molecules as well as fragments thereof, such as, for example, Fab and F(ab′)₂, which are capable of binding antigen. Fab and F(ab′)₂ fragments lack the Fc fragment of intact antibody, clear more rapidly from the circulation, and may have less non-specific tissue binding than an intact antibody [Wahl et al., J. Nucl. Med. 24: 316-325, (1983)]. It will be appreciated that Fab and F(ab′)₂ and other fragments of the antibodies useful in the present invention may be used for the detection and quantitation of the ligand for the complex of the invention, according to the methods disclosed herein for intact antibody molecules. Such fragments are typically produced by proteolytic cleavage, using enzymes such as papain (to produce Fab fragments) or pepsin (to produce F(ab′)₂ fragments).

An antibody is said to be “capable of binding” a molecule if it is capable of specifically reacting with the molecule to thereby bind the molecule to the antibody. The term “epitope” is meant to refer to that portion of any molecule capable of being bound by an antibody that can also be recognized by that antibody. Epitopes or “antigenic determinants” usually consist of chemically active surface groupings of molecules such as amino acids or sugar side chains, and have specific three-dimensional structural characteristics as well as specific charge characteristics.

An “antigen” is a molecule or a portion of a molecule capable of being bound by an antibody, which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

The antibodies, including fragments of antibodies, useful in the present invention, may be used to quantitatively and/or qualitatively detect the ligand for the complex of the present invention in a sample. This can be accomplished by immunofluorescence techniques employing a fluorescently or color-labeled antibody coupled with light microscopic, flow cytometric, or fluorometric detection.

Another specifically preferred embodiment relates to the antibodies of the invention conjugated to a detectable moiety. One of the ways in which an antibody in accordance with the present invention can be detectably labeled is by linking the same to an enzyme and used in an enzyme immunoassay (EIA). This enzyme, in turn, when later exposed to an appropriate substrate, will react with the substrate in such a manner as to produce a chemical moiety which can be detected, for example, by spectrophotometric, fluorometric or by visual means. Enzymes which can be used to detectably label the antibody include, but are not limited to, malate dehydrogenase, staphylococcal nuclease, delta-5-steroid isomerase, yeast alcohol dehydrogenase, α-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholin-esterase. The detection can be accomplished by colorimetric methods, which employ a chromogenic substrate for the enzyme. Detection may also be accomplished by visual comparison of the extent of enzymatic reaction of a substrate in comparison with similarly prepared standards.

Detection may be accomplished by using any of a variety of other immunoassays. For example, by radioactive labeling the antibodies or antibody fragments, it is possible to detect receptor tyrosine phosphatase (R-PTPase) through the use of a radioimmunoassay (RIA). A good description of RIA may be found in Laboratory Techniques and Biochemistry in Molecular Biology, by Work, T. S. et al., North Holland Publishing Company, NY (1978) with particular reference to the chapter entitled “An Introduction to Radioimmune Assay and Related Techniques” by Chard, T., incorporated by reference herein. The radioactive isotope can be detected by such means as the use of a g counter or a scintillation counter or by autoradiography.

It is also possible to label an antibody in accordance with the present invention with a fluorescent compound, fluorescence emitting metals, a chemi-luminescent compound or a bioluminescent compound.

In a fourth aspect, the invention relates to a composition for the treatment of a pathological condition. This composition comprises as active ingredient a complex according to the invention, in which the active segment is a cytotoxic moiety and/or an Ig fragment and pharmaceutically acceptable carriers.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

The composition of the invention may be used in the treatment of viral infections by, for example, Influenza virus, human immunodeficiency virus, Epstein-Barr virus, cytomegalovirus, Vaccinia virus, MVM, ECMV and Herpes virus.

The composition of the invention may also be used for treating a cancer disease such as melanoma, carcinoma, sarcoma and lymphoma, preferably melanoma.

An alternative aspect of the present invention relates to a diagnostic composition for detecting the presence of abnormal cells in a sample, comprising a complex of the invention in which the active segment is a detactable imaging moiety such as a paramagnetic, radioactive or fluorogenic agent or moiety.

Another aspect of the present invention relates to a method for treating a pathological condition in a subject comprising the step of administering a pharmaceutically effective amount of a therapeutic agent to the subject, wherein said therapeutic agent comprises a complex or composition of the invention.

The pathological condition may be a viral infection caused by any one of Influenza virus, human immunodeficiency virus, Epstein-Barr virus, cytomegalovirus, Vaccinia virus, MVM, ECMV and Herpes virus.

Alternatively, the method of the invention may be used for treating a malignant disease such as melanoma, carcinoma, sarcoma and lymphoma, and preferably melanoma.

The method of the invention preferably employs a complex of the invention in which the active segment comprises an Ig fragment, particularly the Fc portion of an Ig molecule denoted by the amino acid sequence substantially as denoted by SEQ ID NO: 5. Alternatively, the method of the invention employs a complex in which the active segment comprises a cytotoxic moiety. This cytotoxic moiety may be selected from cytotoxins or anticellular agents capable of killing and/or suppressing the growth and/or cell division of said target cell. More specifically, the cytotoxin or anticellular agent may be a synthetic toxin or a plant-, fungus-, or bacteria-derived toxin. For example, the toxin may be an A chain toxin, ribosome inactivating protein, α-sarcin, aspergillin, restrictocin, ribonuclease, diphtheria toxin, Pseudomonas exotoxin, an endotoxin or the lipid A moiety of an endotoxin.

In addition to the method of treatment, the present invention encompasses ex vivo treatment, by which cancerous cells or otherwise defective cells which express any one of NKp46, NKp44 and NKp30 on their surface are treated with the complex of the invention. In such ex vivo protocols, the biological sample may be drawn from the body of the subject, such as a human subject. The sample may be of blood, bone marrow cells, or similar tissues or cells from an organ afflicted with a cancer. Methods for obtaining such samples are well known to the skilled workers in the fields of oncology and surgery. They include sampling blood in well-known ways, or obtaining biopsies from the bone marrow or other tissue or organ. The cancer cells (or virus-infected cells) contained in the sample may be effectively eliminated due to the cytotoxic activity of the complex of the invention. The sample may then be returned to the body of the subject from which it was obtained.

As used herein, “effective amount” means an amount necessary to achieve a selected result. For example, an effective amount of the composition of the invention useful for the treatment of said pathology.

In a preferred embodiment, the method of the invention is intended for treating a mammalian subject, preferably, a human. Therefore, by “patient” or “subject in need” is meant any mammal for which gene therapy is desired, including human bovine, equine, canine, and feline subjects, preferably, human patient.

For the in vivo treatment in accordance with the invention, the complex or compositions of the invention can be administered in a variety of ways. By way of non limiting example, the cells may be delivered intravenously, or into a body cavity adjacent to the location of a solid tumor, such as the intraperitoneal cavity, or injected directly into or adjacent to a solid tumor. Intravenous administration, for example, is advantageous in the treatment of leukemias, lymphomas, and comparable malignancies of the lymphatic system, as well as in the treatment of viral infections.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacterium and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

The complex and compositions of the present invention may be administered directly to the subject to be treated or, depending on the size of the compound (cytotoxic or imaging moiety), it may be desirable to conjugate them to carrier proteins such as ovalbumin or serum albumin prior to their administration. Therapeutic formulations may be administered in any conventional dosage formulation. Formulations typically comprise at least one active ingredient, as defined above, together with one or more acceptable carriers thereof.

Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy.

An alternative aspect of the present invention relates to a method for the diagnosis and imaging of pathologies, specifically tumors. This method comprises the steps of introducing an imaging agent into the blood stream of a subject, and detecting and quantitating the binding of the imaging agent to any one of NKp46, NKp44 or NKp30 ligands expressed on a diseased target cell. The imaging agent comprises a complex of the invention in which the active segment comprises an imaging moiety that can be, for example, a paramagnetic, radioactive or fluorogenic agent.

In a specifically preferred embodiment, the diagnostic method of the invention may be used for the diagnosis of pathological conditions such as tumors, e.g. melanomas, carcinomas, sarcomas and lymphomas.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Materials and Methods

Cells and Viruses

Cell Lines:

721.221—class I MHC-negative human EBV-transformed B cell line.

1106mel—class I MHC-negative human melanoma cell line.

293T—adenovirus-transformed, SV-large T antigen-transfected, human fibroblast kidney cell line.

NK cells (lines and clones) were isolated from peripheral blood lymphocyte (PBL) using the human NK cell isolation kit and the autoMACS instrument (Miltenyi Biotec Inc), NK cells were kept in culture as previously described [Mandelboim, O., et al. J. Exp. Med. 184:913-922 (1996)].

Viruses:

Sendi virus (SV) a mouse paramyxovirus and the Influenza virus (IV) A/PR/8/34 (H1N1) were purchased from Spafas (Preston City, Conn., USA).

Monoclonal Antibodies

Sendi virus specific mAb were previously described [Peterhans, E., et al., Virology 128:366-376 (1983); Yewdell, J. W. et al., J. Immunol. 128:2670-2675 (1982)].

Influenza virus specific mAb were previously described [Yewdell, J. W., et al., J. Virol. 48:239-248 (1983)].

Anti-CD99 mAb 12E7 is a kind gift from A. Bernard (Hopital de L'Archet, Nice, France).

The anti-KIR2DL1 (NKAT1) mAb HP3E4 is a kind gift from Dr. Lopez-Botet (Hospital de la Princesa, Madrid, Spain). The hybridoma-producing mAb 3G8 was kindly given by Jay Unkeless (Mt. Sinai School of Medicine, New York, USA). The pan anti-class I mAb 147 was purchased from ExBio (Czech Republic).

The hybridoma-producing mAb 3G8 was kindly given by Jay Unkeless (Mt. Sinai School of Medicine, New York, USA).

General Methods in Molecular Biology:

Standard molecular biology techniques known in the art and not specifically described were generally followed as in Maniatis et al., Molecular Cloning: A Laboratory Manual, Cold Spring Harbor Laboratory, New York (1989, 1992).

Generation of mAb that Recognize 721.221 Cells Infected with SV

SV-infected 721.221 cells (5×10⁶) were washed and injected intraperitoneally three times into BALB/c mice at 14 day intervals. Sera were harvested from immunized mice and tested for the presence of antibodies against infected cells. Mice producing such antibodies were re-boosted, spleens harvested 4 days later, and the splenocytes fused with SP2/0 cells as previously described [Porgador, A., et al., Immunity 6:715-726 (1997)].

Supernatants from wells containing growing cell fusions were first screened by ELISA for binding 721.221, SV-infected 721.221 or 1106mel cells. Supernatants positive for infected 721.221 and/or 1106mel were then screened by flow cytometry for blocking of NKp46-Ig binding to SV-infected 721.221 cells.

Hemagglutination Inhibition Test

In microtitration plates 1 hemagglutination unit (HAU) of IV was incubated for 30′ at 4° C. in 50 μl PBS with serial two-fold dilutions of NKp46-Ig. 50 μl of 1% packed sheep RBC suspension was then added and the pattern of hemagglutination counted after 30 min at room temperature.

Cytotoxicity Assays

The cytotoxic activity of NK lines and clones against the various targets was assessed in 5-hr ³⁵S-release assays as previously described [Porgador, A., Proc Natl Acad Sci. USA 94:13140-13145 (1997)].

In experiments where mAb were included, NK cells were first incubated with 50% human serum (to prevent binding of the mAb to the various Fc receptors expressed on the surface of human NK cells) and washed. The final mAb concentration was 20 μg/ml or 1:100 dilution in cases where the mAbs are present in sera from hybridoma bearing mice. In all experiments shown, the spontaneous release was less than 25% of maximal release. Each point represents the average of duplicate values. The range of the duplicates was always within 5% of their mean.

Ig-Fusion Proteins

The generation of CD16-Ig fusion protein was previously described [Mandelboim, O. et al., Proc. Natl. Acad. Sci. USA 96:5640-5644 (1999)].

Sequences encoding the extracellular portions of Sequences encoding the extracellular portions of NKp30 (accession number AJ223153), NKp44 (accession number NM_{—}004828), NKp46 (isoform used has accession number AJ006121), KIR-1 (accession number L41267) and NKAT-8 (NM_{—}012314) were amplified by PCR from cDNA isolated from human NK clones. These PCR-generated fragments were cloned into the mammalian expression vector containing the Fc portion of human IgG1 and Ig-fusion proteins were produced as described [Mandelboim, O. et al., (1999) ibid.]. Sequencing of the constructs confirmed that cDNA of all Ig-fusion proteins were in frame with the human Fc genomic DNA and were identical to the reported sequences.

All Ig-fusion proteins used in this work migrate as a single band on standard non-reduced SDS-PAGE gels and each was regularly assayed by SDS-PAGE to ensure the proteins had not degraded.

FACS staining procedure with Ig-fusion proteins was previously described [Mandelboim, O. et al., (1999) ibid.].

Production of Anti-NKp46-Ig and NKp44-Ig Serum

BALB/c mice were injected in their foot pads with 40 μg of NKp46-Ig, NKp44-Ig or KIR-1-Ig fusion proteins emulsified in CFA. Six weeks later mice were boosted and sera were harvested 12 days later.

For control sera, mice were immunized as above with PBS emulsified in CFA. Sera were tested for the NKp46 and NKp44 antigen specificity on YTS, KIR-1-transfected YTS cells and various NK lines and clones.

The anti-NKp44 and 46 serum was used at 1:100 dilution, a concentration at which binding was saturated as measured by flow cytometry. In agreement with the pattern of expression of NKp44 and NKp46 reported [Cantoni C, C. et al., J. Exp Med. 189: 787-796 (1999), Pessino A, S. et al., J. Exp. Med. 188: 953-960 (1998)], only NK cells were positively stained with the anti-NKp44 and anti-NKp46 serums and control cells 721.221, RPMI 8866, Jurkat and others, remained unstained. All NK cells (6 lines and more than 150 clones) were stained to various degrees with this serum. In agreement with the pattern of expression of NKp44 reported [Cantoni C, C. et al. ibid (1999)], expression of NKp44 was detected only on the surface of activated NK cells. In addition, re-directed lysis of P815 cells could be induced when the cells were coated with the anti-NKp44 and 46 serum and incubated with various NK lines and clones. The anti-NKp44 and 46 serum partially blocked the lysis of several non-infected target cells, even including lysis of non-infected 1106mel and 293T cells at high E:T ratios (greater than 20:1). The presence of cellular ligands to other NK triggering receptors [as reviewed in Bottino, C., et al., Hum. Immunol. 61: 1-6 (2000)] might explain the partial blocking.

Blocking of Binding of NKp46-Ig and NKp44-Ig Fusion Proteins to Virus-Infected Cells

Cells (721.221 and 1106mel) were infected either with 100 μl of SV supernatant (for 721.221), or with 1000 u/ml of IV (for 1106mel). After overnight incubation, infected cells were washed and incubated on ice with the various mAbs, for 1 h. Next, cells were washed and assayed for staining with 10 μg of the appropriate Ig-fusion protein as previously described [Mandelboim, O., et al., (1999) ibid.].

Blocking of NKp44-Ig Binding Using Purified HA.

Ten micrograms of various Ig-fusion proteins were incubated for two hrs on ice with 40 μg of purified HA protein [Brand, C. M. and Skehel, J. J. Nat. New Biol. 238:145-147 (1972)] at final volume of 100 μl PBS-BSA-Azid. These mixtures were then incubated with cells for 2 hrs on ice and stained for the presence of Ig-fusion proteins using the same staining procedures as previously described [Mandelboim, O., P. et al., ibid (1999)].

Transient Transfection

293T cells were either transiently transfected with a control PCDNA3 plasmid (293T/MOCK) or with a cDNA encoding for HN of the Sendai Virus (293T/pca-svhn) using the Fugene transfection reagent (Boehringer Mannheim).

### Example 1

Detection of NKp30 NKp44, NKp46 and CD]6 Ligands.

The role of NKp30, NKp44 NKp46 and CD16 in NK recognition was studied by producing fusion proteins in which extracellular domains of NKp30, NKp44, NKp46 and CD16 are fused to the Fc portion of human IgG1. The extracellular domain of CD99 fused to the Fc of the human IgG1 DNA was used as control. These constructs were transiently transfected into COS-7 cells and secreted fusion proteins were purified on a protein G column. The Ig-fusion proteins were incubated with the various target cells and analyzed for binding by indirect immunostaining as previously described [Mandelboim, O., et al.,. ibid (1999)]. In general, among all the Ig-fusion proteins tested, the most efficient binding was observed with the NKp44-Ig fusion protein (Table 1). The control CD99-Ig fusion protein did not bind to any of the target cells tested. Previous reports suggested that NKp46, together with the NKp44 activating receptor but not the CD16 receptor [Mandelboim, O., et al., ibid. (1999)], are involved in the lysis of class I negative 721.221 cells [Cantoni C, C. et al., ibid (1999), Biassoni R, A. et al., Eur J Immunol. 29: 1014-1020 (1999), Pessino A, S. et al., J. Exp. Med. 188: 953-960 (1998); Sivori S, D. et al., Eur J Immunol. 29: 1656-66 (1999)].

Indeed, little staining of 721.221 cells was observed when cells were incubated either with the NKp30-Ig, NKp44-Ig or NKp46-Ig fusion proteins and no staining was observed when the 721.221 cells were incubated with the CD16-Ig fusion protein (Table 1). Similar results were obtained with another EBV transformed B cell line, RPMI 8866. These results indicate that the low binding of NKp30-Ig, NKp44-Ig and NKp46-Ig, suggesting low ligands expression for all of these proteins, is either nevertheless enough to cause lysis of these cells, or alternatively suggests the existence of another lysis ligand for other lysis receptors different from NKp44, NKp46 or CD1 6. Binding of NKp30-Ig, NKp44-Ig, NKp46-Ig or CD16-Ig to large T antigen transfected 293T kidney cell line, to the 1106mel-melanoma cell line and to the monkey COS-7 cell line, was observed (Table 1). Indeed, all of these target cells are sensitive, to various degrees, to NK cell mediated killing [Mandelboim, O., P. et al., ibid. (1999)]. The presence of ligands for NKp30 NKp44, NKp⁴⁶ and CD16 on COS-7 cells suggests that the ligands for these receptors might be conserved among some primates. Little staining of NKp30 and NKp44-Ig fusion proteins to LB33MELA1 melanoma cells line was observed (Table 1); a cell line that can not be killed by NK cells (data not shown). Finally, little or no binding of either NKp30-, NKp44-, NKp46- or CD16-Ig fusion proteins was observed, either to the mouse p815 cells or to PBL derived from healthy donors (Table 1), thus indicating foremost that the ligands for the lysis receptors might be different between human and mouse and secondly that “normal” cells, derived from PBL, at least, do not express the lysis ligands for NKp30 NKp44, NKp46 and CD16.

TABLE 1
Staining of different target cells with various Ig-fusion proteins

| | No | | | NKp30- | NKp44- | NKp46- |
|---|---|---|---|---|---|---|
| Cells | protein | CD99-Ig | CD16-Ig | IG | Ig | Ig |
| 721.221 | 3 | 4 | 3 | 11 | 9 | 11 |
| RPMI | 3 | 4 | 4 | 10 | 9 | 9 |
| 8866 | | | | | | |
| 293T | 3 | 4 | 12 | 57 | 125 | 13 |
| I106mel | 3 | 4 | 9 | 60 | 99 | 11 |
| LB33ME | 3 | 4 | 4 | 8 | 7 | 4 |
| LA1 | | | | | | |
| COS | 6 | 4 | 16 | 91 | 100 | 14 |
| P815 | 2 | 2 | 3 | 5.2 | 3 | 4 |
| PBL | 2 | 2 | 2 | 57 | 2 | 2 |

Cells were incubated with various Ig-fusion proteins as described in the experimental procedures and stained with PE-conjugated goat anti-human Fe. MFI indicates Median Fluorescence Intensity; MFI numbers were rounded to the nearest whole numbers. Results are representative of two independent experiments.

### Example 2

NKp46-Ig Fusion Protein Binds to Virus Infected Cells

As previously published [Trinchieri et al., Adv. in Immunol. 47:187-376 (1989)], NK cells can effectively lyse virus-infected cells, therefore the question whether infection with Sendai virus (SV) (a mouse paramyxovirus) increased the binding of NKp46-Ig was next tested. Remarkably, a 10-fold increase in the staining by NKp46-Ig was observed (Table 2). This effect is specific for NKp46 since SV infection did not alter the binding of other NK receptor Ig-fusion proteins tested (CD16-Ig, KIR-1-Ig, or NKAT-8-Ig—data not shown).

TABLE 2
Anti-hemagglutinin antibodies inhibit the NKp46-Ig binding to
Sendai virus-infected cells.

| | | | | MFI NKp46-Ig | |
|---|---|---|---|---|---|
| | | MFI binding of mAb | | binding | |
| | mAb | | 721.221 | | 721.221 |
| | specificity | 721.221 | Sendai | 721.221 | Sendai |
| No mAb | — | 5 | 5 | 6 | 74 |
| TC-9A1 | Anti-Fusion | 5 | 82 | 6 | 63 |
| TC-1D6 | Anti-HN | 7 | 141 | 7 | 14 |
| TC-9C1 | Anti-HN | 13 | 179 | 7 | 18 |
| 135.7 | Anti-HN | 5 | 95 | 6 | 22 |
| 721.221 cells (10⁶/ml) were incubated overnight with 100 μl/ml of SV-containing supernatant. Cells (infected or uninfected) were washed, incubated with various mAbs and stained either with FITC-labeled goat anti-mouse Ig, or with the NKp46-Ig fusion protein followed by PE-conjugated goat anti-human Fc. MFI indicates Median Fluorescence Intensity; MFI numbers were rounded to the nearest whole numbers. | | | | | |
| #Background staining of SV-infected 721.221 and 721.221 cells with the PE-conjugated anti-human Fc was 4 and 2, respectively. Results are representative of five independent experiments. Similar results were obtained with another B cell line, RPMI 8866. | | | | | |

Infection with Different Viruses

To find out whether the observed binding of the NKp46-Ig fusion protein to SV infected 721.221 cells was virus or cell specific, similar infection experiments were performed using the A9 fibroblasts infected with different viruses. As shown in FIG. 1, significant increase in the NKp46-Ig binding was observed when uninfected A9 cells were compared to the same cells infected with EMCV, MVM, Adeno virus or Vaccinia virus.

### Example 3

Binding of NKp46-Ig to the SV HN Glycoprotein

To identify the putative NKp46-ligand on SV-infected 721.221 cells, mice were immunized with the SV-infected 721.221 cells, and spleen derived B cell hybridoma supernatants were screened for increased staining of virus-infected cells relative to non-infected cells. The supernatants of one of the hybridoma clones tested (135.7) efficiently blocked the binding of NKp46-Ig to SV infected cells (Table 2). Therefore, mAb 135.7 may recognize either proteins encoded by SV or host cells.

ELISA assays using SV as immunoadsorbent indicated that mAb 135.7 recognizes a viral gene product (data not shown).

SDS-PAGE analysis of 135.7-reactive proteins recovered from detergent lysates of ¹²⁵I-cell surface labeled SV-infected 721.221 cells, revealed that 135.7 binds a protein with an apparent M.W. of 70 kDa, similar to the reported mobility of the HN glycoprotein.

Therefore the question whether well characterized anti-SV mAb also block the binding of NKp46-Ig to SV-infected cells, was next tested. Indeed, as shown in Table 2, NKp46-Ig binding was blocked when infected cells were first incubated with anti-HN mAb TC-1D6 or TC-9C1 but not with mAb TC-9A1 specific for the other major SV glycoprotein, the fusion (F) protein.

### Example 4

The Role of NKp46 Recognition of HN in Lysis of HN Transfected 293T Cells

Despite the HN-dependent binding of NKp46-Ig to SV infected 721.221 cells, detection of an increased susceptibility of these cells to NK mediated lysis associated with SV infection was failed (data not shown), probably because these cells are already very sensitive to NK-mediated lysis. This prevented the direct testing of the role of NKp46 recognition of HN in NK lysis.

However, this was possible using 293T cells that were transiently transfected with a plasmid encoding HN. Forty-eight h after transfection, cell surface HN expression was confirmed using the TC-1D6 mAb (FIG. 2A) or 135.7 (not shown).

Importantly, as shown in FIG. 2A, transfection resulted in a two-fold increase in NKp46-Ig staining, without enhancing the staining with other Ig-fusion proteins—KIR-1-Ig, NKAT-8-Ig or CD16-Ig. Moreover, NK-GAL, a NK line derived from healthy donor PBL, lysed HN-transfected 293T cells at least 4-fold more efficiently than non-transfected or mock-transfected cells (FIGS. 2B,C).

Pre-incubation of NK GAL with a mouse antiserum raised against NKp46-Ig, resulted in inhibition of the increased killing of HN-transfected 293T cells, while incubation with a control serum had little effect (FIG. 2B). The same experiment revealed that each of three HN-specific mAb could block NK mediated lysis, while a control mAb specific for CD99 had no significant effect (FIG. 2C).

### Example 5

NKp46 is Required for the Recognition of HA-Expressing Cells by NK Cells

One of the hallmarks of NK recognition is its lack of antigen specificity. Having shown that NKp46 interacts with the SV HN both physically and functionally in NK mediated lysis, the inventors next tested whether it could interact with the Influenza virus (IV) HA. IV-infection of 1106mel cells (a class I deficient cell line) resulted in a four-fold increase in NKp46-Ig binding (Table 3). As above, the specific nature of the enhanced binding is shown by the constant binding of other Ig-fusion proteins (data not shown).

Importantly, the increased NKp46-Ig binding was completely, or partially blocked, respectively, by the HA specific mAb H28-E23 and H17-L2 (Table 3). In contrast, HN specific mAb TC-1D6, TC-9C1 or 135.7 had no effect on binding (data not shown).

Next, 1106mel cells were infected with IV. As shown in FIG. 3A, this infection enhances NK GAL mediated lysis, and the enhanced killing is blocked by pre-incubation of cells with anti-NKp46 serum but not with control serum or mAb 12E7 and 3G8 specific for CD99 and CD16 respectively.

Incubation of IV-infected 1106mel cells with mAb H28-E23 resulted in complete inhibition of the increased lysis, whereas H17-L2 had a partial inhibitory effect, mirroring the results of the blocking experiment (FIG. 3B, Table 3).

TABLE 3
Anti-hemagglutinin antibodies inhibit the NKp46-Ig binding to
Influenza virus-infected cells.

| | | MFI binding of mAb | | MFI NKp46-Ig binding | |
|---|---|---|---|---|---|
| | mAb | | 1106mel | | 1106mel |
| | specificity | 1106mel | Influenza | 1106mel | Influenza |
| No mAb | — | 18 | 22 | 112 | 441 |
| H28-E23 | Anti-HA | 18 | 2016 | 100 | 63 |
| H17-L2 | Anti-HA | 11 | 2100 | 110 | 145 |
| NA2-1C1 | Anti-NA | 10 | 2000 | 109 | 980 |
| 1106mel cells (10⁶/ml) were incubated O.N. with 1000 u/ml of Influenza virus. Cells (infected or uninfected) were washed, incubated with various mAb, and stained either with FITC-conjugated goat anti-mouse Ig, or with the NKp46-Ig fusion protein followed by PE-conjugated goat anti-human Fc. MFI indicates Median Fluorescence Intensity; MFI numbers were rounded to the nearest whole numbers. | | | | | |
| #Background staining of IV-infected 1106 and 1106 cells with the PE-conjugated anti-human Fc was 7 and 6, respectively. Results are representative of eight independent experiments. | | | | | |

The recognition of IV-infected 1106mel cells by clones prepared from NK GAL was examined by limiting dilution. All 28 clones tested were positively stained with the anti-NKp46 serum. Twenty-one of the clones exhibited enhanced recognition of IV-infected cells relative to uninfected cells (FIG. 3C); pre-incubation of all 21 clones with the HA-specific mAb H28-E23 completely inhibited the IV-enhanced lysis (data not shown). Pre-incubation of NK clones with anti-NKp46 serum completely inhibited the IV-enhanced lysis of 13 of these clones, (e.g. clone 6, FIG. 3C), while 6 of the clones were partially inhibited (e.g. clone 15, FIG. 3C). The average MFI staining with anti-NKp46 serum for these two groups was 28.9 and 32.8 respectively. Two clones demonstrating enhanced lysis of IV-infected cells were not affected by the NKp46 antiserum (e.g. clone 17, FIG. 3C). The average MFI for anti-NKp46 staining for this group of two clones was 17.5.

Finally, no IV-associated enhancement in lysis was observed in 7 of the clones tested, (e.g. clone 5, FIG. 3C) and the MFI for anti-NKp46 staining of this group was 31.3. Similar results were obtained when the same NK clones were tested with HN transfected 293T cells (data not shown).

NK clones were also generated from other NK lines that express lower amount of the NKp46 receptor (for example, an NK line with MFI of NKp46 staining of 15.5 as compared to MFI of 41.4 in NK GAL). Inhibition (either partial or complete) of the enhancement of IV-infected 1106mel lysis was observed in about third of the clones generated from this NK line, and the extent of lysis correlated with NKp46 staining (data not shown).

These findings indicate first, that NKp46 is required for the recognition of HA- and HN-expressing cells by a substantial subset of NK cells, and second, that other populations of NK cells can lyse these cells in an NKp46 independent manner.

### Example 6

Involvement of Sialic Acid in the Interaction of NKp46 with HA and HN

SV HN and IV HA both recognize terminal N-acetyl neuraminic acid residues (sialic acids) attached to Gal, suggesting a common mechanism for binding to NKp46. The involvement of sialic acid in the interaction of NKp46 with HA is indicated by a number of findings. First, NKp46-Ig is able to completely block IV-mediated agglutination of sheep erythrocytes at a protein concentration as low as 2 μM. Second, pre-incubation of TV-infected cells with a mAb (NA2-1C21) that blocks the enzymatic activity of IV neuraminidase (the other major IV glycoprotein expressed on the surface of infected cells and virions) significantly enhances NKp46-Ig binding to IV infected cells (Table 3). Finally, and most directly, treatment of NKp46-Ig with bacterial neuraminidase reduced its binding to IV-infected cells without reducing its binding to uninfected cells (FIG. 4).

Inasmuch as desialylation often increases interactions by reducing negative charge repulsion of receptor-ligand pairs [Varki, A., et al., FASEB J. 11:248-55 (1997)], this strongly supports the direct interaction of NKp46 with the sialic binding site of HA.

These findings can be interpreted to indicate that NKp46 binds to target cells via two types of ligands: the first based on interaction of NKp46-associated sialic acid with viral sialic acid receptors, the second on a sialic acid-independent interaction with undefined cellular ligands. The former is clearly responsible for the enhanced killing of IV-infected cells by the NK cells that were studied. The contribution of the second interaction to NK activation remains to be established, and probably varies depending on the nature of the ligands expressed by the target cells and other factors as well. Whether the interaction of NKp46 with viral HA is sufficient for triggering or also requires interaction with other cellular ligands remains an important question for future studies.

The existence of NK clones that recognize IV-infected cells in a NKp46 independent manner (e.g. NK GAL clone 17, FIG. 3C) suggests the existence of other lysis receptors involved in the recognition of virus infected cells. These receptors also probably recognize HA, since the enhanced lysis associated with virus infection is completely blocked by anti-HA mAb. It is possible that the triggering of these receptors is also based on the interaction of the activating receptor with sialic acid. Given that members of at least 7 virus families utilize sialic acid as a receptor for virus entry into host cells, this suggests a general strategy for NK cell recognition of a substantial subset of viruses.

### Example 7

Up-Regulation of NKp44-Ig Binding to SV-Infected 721.221 Cells is Blocked by Anti-HN mAb.

As was described herein before, the viral HA was identified as a ligand for NKp46 receptor. To test whether the NKp44 receptor can also bind to viral HA, 721.221 cells were infected with SV and tested for increased binding of NKp44-Ig. A 10-fold increase in the staining by NKp44-Ig was observed (Table 4). This effect is specific for NKp44-Ig and NKp46 (see table 2 as well), since SV infection did not alter the binding of other NK receptor Ig-fusion proteins tested (NKp30-Ig, CD16-Ig, KIR-1-Ig, or NKAT-8-Ig (data not shown). The NKp44-Ig binding was partially blocked by mAb directed against SV-HN, TC-1D6, TC-9C1 [Peterhans, E., et al., Virology. 128: 366-376 (1983)] or 135.7, but not with mAb TC-9A1 [Peterhans, E., et al., ibid. (1983)] specific for the other major SV glycoprotein, the fusion (F) protein (Table 4). This suggests that NKp44-Ig can interact with HN from SV.

Elevation of NKp44-Ig Binding to 293T Cells Transfected with the SV-HN cDNA

To further demonstrate direct binding of NKp44-Ig to Sendai HN, 293T cells were transiently transfected with an expression plasmid encoding Sendai HN cDNA (pca-svhn, a kind gift from Dr. Allen Portner). Forty-eight hrs after transfection, efficient HN staining was observed when using the anti-HN mAb (TC-1D6, FIG. 5). Similar levels of expression were also observed when stained with 135.7 mAb, but not with control mAb (anti-HA of Influenza, data not shown). Importantly, a two-fold increase in NKp44-Ig staining of the pca-svh transfected 293T cells was also observed (FIG. 5), confirming the specificity of the increase to be a ligand for NKp44. No change in the staining of all tested cells was observed using other Ig-fusion proteins (KIR-1-Ig, NKAT-8-Ig or CD16).

TABLE 4
Anti-hemagglutinin antibodies inhibit the NKp44-Ig binding to
Sendai virus-infected cells.

| | | binding of mAb (MFI) | | NKp46-Ig binding (MFI) | |
|---|---|---|---|---|---|
| | mAb | | 721.221 | | 721.221 |
| | specificity | 721.221 | Sendai | 721.221 | Sendai |
| No | — | 4 | 7 | 11 | 108 |
| mAb | | | | | |
| TC-9A1 | Anti-Fusion | 4 | 72 | 12 | 91 |
| TC-1D6 | Anti-HN | 6 | 139 | 11 | 64 |
| TC-9C1 | Anti-HN | 5 | 124 | 12 | 51 |
| 135.7 | Anti-HN | 6 | 108 | 13 | 55 |
| 721.221 cells (10⁶/ml) were incubated overnight with 100 μl/ml of Sendai virus-containing supernatant. Cells (infected or uninfected) were washed, incubated with various mAb and stained, either with FITC-labeled goat anti-mouse Ig or with the NKp46-Ig fusion protein, followed by PE-conjugated goat anti-human Fc. MFI indicates Median Fluorescence Intensity; MFI numbers were rounded to the nearest whole | | | | | |
| #number. Background staining of Sendai virus-infected 721.221 and 721.221 cells with the PE-conjugated anti-human Fc was 4 and 3, respectively. Results are representative of four independent experiments. | | | | | |

### Example 8

SV Infection of 721.22/Cw6 Cells Resulted in the Abrogation of the Inhibition Mediated by NK Clones Expressing High Levels of NKp44 Proteins

The possibility that infection of cells with SV would enhance NK-mediated lysis was next investigated. No change in the NK mediated killing was observed between the SV-infected 721.221 cells and non-infected cells (data not shown). Several explanations may account for this phenomena. Among these is the observation that NK cells efficiently lyse 721.221 cells, and it is therefore possible that the addition of another lysis ligand (SV hemagglutinin) will not result in increased killing. Therefore, the inventors next tested whether any change in the killing pattern of NK clones will be observed when 721.221 cells expressing class I MHC proteins will be infected with SV. NK clones were prepared from various donors and were first screened for inhibition of lysis mediated by 721.221 cells transfected either with HLA-Cw3, -Cw4, -Cw6 or -Cw7 class I MHC proteins. The generation of these transfectants was described by O. Mandelboim [Mandelboim, O., et al., J. Exp. Med. 184: 913-922 (1996)]. NK clones that were found to be inhibited by 721.221 cells expressing class I MHC proteins were next tested against the same target cells infected with SV. SV infection of 721.221 cells resulted in the abrogation of the inhibition and consequently lysis by about 75% of the NK clones tested. All of these clones expressed high levels of both NKp44 and NKp46 proteins (data not shown). One representative clone is seen in FIG. 6. NK clone 66 is inhibited by 721.221 cells expressing Cw6 (721.221/Cw6). Abolishment of the inhibition was observed when NK clone 66 was incubated with the anti-KIR2DL1 mAb HP3E4 but not when the cells were incubated with the control mAb 12E7 (FIG. 6A). Reversal of the inhibition was also observed when 721.221/Cw6 cells were infected with SV. The inhibition was made possible due to the interaction of the KIR2DL1 receptor with HLA-Cw6 as blocking of the inhibition was observed when721.221/Cw6 cells were incubated with the pan anti-class I mAb 147 (FIG. 6B). The reversal of the inhibition was dependent on the expression of HA on the infected cells as inhibition was restored when the infected cells were incubated with the anti-HA mAb 135.7 (FIG. 6B). Similar results were obtained when NK clone 66 was incubated with 721.221/Cw4 cells or with other NK clones expressing the KIR2DL2 receptor inhibited by 721.221/Cw3 or 721.221/Cw7 (data not shown). About 25% of the NK clones tested that expresses the NKp44 protein at low levels showed no change in the inhibition pattern when target cells were infected with SV. Thus, the interaction of NKp44 with the HA of SV is probably needed to overcome the inhibition mediated by SV-infected target cells expressing class I MHC protein.

### Example 9

Purified HA Blocks the Binding of the Fusion Proteins NKp46-Ig Binding and NKp44-Ig to Virus Infected Cells

The involvement of both NKp44 and NKp46 in the killing of the infected 721.221 cells (wild type and transfectants) can no be directly tested in this system using the anti-NKp44 and NKp46 serums as the lysis of 721.221 cells is NKp44 and NKp46 dependent [Cantoni C, C. et al., ibid (1999), Biassoni R, A. et al., ibid (1999), Pessino A, S. et al., ibid (1998), Sivori S, D. et al., ibid (1999)]. Therefore, the possibility that the NKp44-Ig fusion protein can bind the hemagglutinin of other viruses using different cell types was next tested. 1106mel cells (a cell line which is only moderately lysed by CD16 positive human NK cells [Mandelboim, O., et al., ibid (1999)] were infected with IV and stained for elevated NKp44-Ig staining. Increased NKp44-Ig staining (about four fold) was observed when the 1106mel cells were infected with IV (Table 5). This NKp44-Ig staining was specific, as no increase in the binding of other Ig-fusion proteins, including NKp30 CD16-Ig, KIR-1-Ig and NKAT-8-Ig, to the IV-infected 1106mel cells was observed (data not shown). Importantly, the increased NKp44-Ig binding was completely blocked by H28-E23 mAb and H17-L2 mAb (Table 5). Both mAb are directed against the HA of Influenza virus. The addition of mAb directed against the HN from SV (TC-1D6, TC-9C1 and 135.7) had no effect (data not shown).

To find out whether purified HA can block the binding of NKp44 or NKp46 to infected cells, ten micrograms of various Ig-fusion proteins were incubated with 40 μg of purified HA protein at final volume of 100 μl in PBS-BSA-Azid, for two hrs on ice as described elsewhere [Brand, C. M et al. Nat. New Biol. 238:145-147 (1972)]. FIG. 7 shows incubation of NKp44-Ig (A) or NKp46-Ig (B) with or without 40 μg purified HA protein (no blocking of NKp46-Ig or NKp44-Ig binding was evident when less than 40 μg of purified HA protein were used). The mixtures were next incubated for 2 hrs on ice with IV-infected or non-infected 1106mel cells and stained with PE-conjugated goat anti-human Fc for the presence of Ig-fusion proteins using the same staining procedures as previously described [Mandelboim, O. et al. ibid (1999)]. FIG. 7 shows one representative experiment out of two performed. Similar results were obtained when SV-infected 721.221 cells were used. These results indicate direct interaction of the viral protein HA and the natural killer cells activating receptors NKp44 and NKp46.

Moreover, direct interactions between the NKp46-Ig and NKp44-Ig and the hamagglutinin protein in an ELISA assay (not shown).

Binding of NKp44-Ig to Cells Infected with Influenza Virus is Dependent on the Sialylation of NKp44

SV HN and IV HA both recognize terminal N-acetyl neuraminic acid residues (sialic acids) attached to Gal, suggesting that binding to NKp44 might occur via the sialic acid residues expressed on NKp44 similarly to the binding of HA to NKp46 (described in Example 6). Indeed, pre-incubation of IV-infected cells with a mAb (NA2-1C21) that blocks the enzymatic activity of IV neuraminidase (the other major IV glycoprotein expressed on the surface of infected cells) significantly enhances NKp44-Ig binding to IV infected cells (Table 5). In addition, NKp44-Ig did not stain target cells infected with measles (data not shown) whose HA does not bind sialic acid [Maisner, A. and Herrler, G. Virology 210: 479-481 (1995)]. Finally, and most directly, treatment of NKp44-Ig with bacterial neuraminidase reduced its binding to IV-infected cells without reducing its binding to uninfected cells (FIG. 8). Treatment of NKp44-Ig did not affect staining of non-infected 1106mel cells, measured by flow cytometry (FIG. 8), nor altered the integrity of the protein tested by SDS/PAGE analysis (data not shown). As desialylation often increases interactions by reducing negative charge repulsion of receptor-ligand pairs [Varki, A., FASEB J. 11: 248-55 (1997)], this strongly supports the direct interaction of NKp44 with the sialic binding site of HA.

TABLE 5
Anti-hemagglutinin antibodies inhibit the NKp44-Ig binding to
Influenza virus-infected cells

| | | binding of mAb (MFI) | | NKp46-Ig binding (MFI) | |
|---|---|---|---|---|---|
| | mAb | | 1106mel | | 1106mel |
| | specificity | 1106mel | Influenza | 1106mel | Influenza |
| No mAb | — | 13 | 13 | 137 | 437 |
| H28-E23 | Anti-HA | 20 | 1290 | 125 | 78 |
| H17-L2 | Anti-HA | 14 | 1670 | 113 | 94 |
| NA2-1C1 | Anti-NA | 13 | 1457 | 109 | 1000 |
| 1106mel cells (10⁶/ml) were incubated O.N. with 1000 u/ml of A/PR/8/34 influenza virus. Cells (infected or uninfected) were washed, incubated with various mAb, and stained, either with FITC-conjugated goat anti-mouse Ig or with the NKp46-Ig fusion protein, followed by PE-conjugated goat anti-human Fc. MFI indicates Median Fluorescence Intensity; MFI numbers were rounded to the nearest whole numbers. | | | | | |
| #Background staining of flu-infected 1106mel and 1106mel cells with the PE-conjugated anti-human Fc was 8 and 6, respectively. Results are representative of three independent experiments | | | | | |

### Example 10

The Enhancement of Lysis of IV-Infected 1106mel Cells is Blocked by Polyclonal mAb to NKp44

The above results suggested that NKp44 can bind the hemagglutinin of both IV and SV. As demonstrated in Example 5, IV infection of 1106mel cells resulted in enhancement of lysis of 1106mel cells that were completely blocked by mAb to HA. However, when anti-NKp46 serum was included in the assays, several killing phenotypes (complete inhibition, partial inhibition or no inhibition) could be observed among the NK clones tested. One possible explanation was the existence of another receptor able to bind HA. The lysis of IV-infected 1106mel was therefore assayed using NK clones and combinations of anti-NKp44 and anti-NKp46 serums. NK clones were prepared from PBL derived from donor MB by limiting dilution. All clones tested (64 in total) were positively stained with both anti-NKp44 and anti-NKp46 serum. Increased killing of the IV-infected 1106mel cells (1106mel/Flu) was observed in 57% of the NK clones tested (FIG. 9). Complete inhibition of the enhancement in 1106mel/Flu lysis was observed in 26% of NK clones tested that were either pre-incubated with anti-NKp46 serum or with the combination of both anti-NKp44 and NKp46 serums (e.g., clone 1, FIG. 9). 14% of the clones tested showed partial inhibition when both anti NKp44 and NKp46 serums were used independently and complete inhibition when both anti-NKp44 and anti-NKp46 serums were combined (e.g., clone 46, FIG. 9). The enhancement in 1106mel/Flu lysis could not be blocked in 17% of the clones tested (for example, clone 8, FIG. 9). Finally, no enhancement in lysis was observed in 43% of the clones tested obtained from this donor, (e.g., clone 4, FIG. 9). The percentage of such cells can vary considerably among different donors, as was showed herein before. Complete inhibition of the enhancement of 1106mel/Flu cells was never achieved when only the anti-NKp44 serum was included in the assay (data not shown). When efficient lysis of 1106mel was observed at higher E:T ratio, incubation of the cells either with anti-CD16 mAb or with anti-NKp44 and NKp46 serum resulted in partial inhibition of lysis (data not shown). When all antibodies were combined, efficient inhibition of 1106mel lysis was observed (data not shown).

Thus, similarly to the NKp46 receptor, NKp44 can bind to the hemagglutinin of both Sendai and Influenza viruses and this binding, results in triggering of NK cell-lysis of the infected cells. The reason for why 43% of the clones tested here showed no increased killing of 1106mel/Flu cells is not completely understood. One possible explanation is that these clones might express receptors to other proteins that are either up- or down-regulated due to the infection and are important in regulating NK killing.

### Example 11

Identification of Domain 2 of the NKp46 as Responsible for Interacting with the Viral HA

The role of different domains of the NKp46 molecule in the interaction with the viral HA was studied by producing different deleted fusion proteins.

The first NKp46 extracellular domain (from a.a. #1-120—as denoted by SEQ ID NO: 26) as well as the second extracellular domain (from a.a. #121-234—as denoted by SEQ ID NO: 22), were fused to the Fc portion of human IgG1, creating the NKp46D1-Ig and the NKp46D2-Ig, respectively. These constructs were transiently transfected into COS-7 cells and secreted fusion proteins were purified on a protein G column. To test whether binding to the viral HA is mediated particularly by one of those two domains, both deletion fusion proteins as well as the control full fusion protein NKp46-Ig and the NKp44-Ig were examined for binding to virus infected cells. Therefore 721.221 cells were infected with SV and tested for increased binding of NKp46D1-Ig or NKp46D2-Ig. About 10-fold increase in the staining by NKp46D2-Ig fusion protein was observed (FIG. 10), whereas SV infection of 721.221 cells did not enhance any significant binding of the NKp46D1-Ig fusion protein. This observed NKp46D2-Ig binding was significantly blocked by the 135.7 mAb directed against SV-HN, but not with mAb TC-9A1 [Peterhans, E., et al., ibid (1983)] specific for the other major SV glycoprotein, the fusion (F) protein or by the control 12E7 antobody (FIG. 10D). These results indicate that the HA interacting portion in NKp46 is the Domain 2. Similar experiments were performed in 1106mel infected with Influenza virus showed that domain 2 of the NKp46 is the domain responsible for the observed interaction with the viral HA (FIG. 11). Thus, NKp46D2 but not NKp46D1 can bind to viral-infected cells and furthermore, this binding can be blocked with anti-HN mAb.

As described in Examples 6 and 9 herein, binding of SV HN and IV HA to NKp46 and NKp44, respectively, occurs via the sialic acid residues expressed on both receptors. In order to find out whether the observed binding of NKp46D2 to the viral proteins is also mediated by sialic residues, the fusion proteins were treated with bacterial neuraminidase prior to their incubation with the infected cells (SV infected 721.221 and IV infected 1106 cells). As shown in FIG. 12, removal of the sialic acid residues using neuroaminidase (NA), significantly reduced the binding capacity of the fusions proteins. These results indicate that the binding of NKp46D2-Ig to the IV-infected 1106mel cells or to the SV-infected 721.221 cells is sialic acid dependent.

### Example 12

Expression of Various Lysis Ligands, and Particularly the NKp30 on Human Melanoma Cells

It has been previously reported that treatment of melanoma patients with Tumor Infiltrated Lymphocytes (TIL) resulted in the emergence of class I MHC loss variants in 40% of the melanoma patients [Restifo, N. P., et al., J. Natl. Cancer Inst. 88:100-108 (1996)]. This is due to downregulation of the β2-microglobulin expression in the tumor cells. The same tumor lines were later shown to be sensitive to various degrees for NK cell mediated killing [Porgador, A., et al., Proc Natl Acad Sci. 94:13140-13145 (1997)]. In addition, it was also shown that one of these melanoma lines, the 11 06mel, was inefficiently killed by many of the CD16-negative NK clones tested [Mandelboim, O., et al., Proc. Natl. Acad. Sci. 96:5640-5644 (1999)].

The role of NKp30, NKp44, NKp46 and CD16 receptors in NK recognition of various melanoma cells deficient in class I MHC expression (except from LB33melA1, used as control) was studied by performing experiments using the NKp30 NKp44, NKp46 and CD16 Ig fusion proteins of the invention. cDNA encoding the extracellular domains of CD99 fused to the human IgG1 DNA was used as control. The Ig-fusion proteins were incubated with the various melanoma cells and analyzed for binding by indirect immunostaining as previously described [Mandelboim, O., ibid. (1999)]. In general, the highest staining of the melanoma cells was observed with the NKp30-Ig and NKp44-Ig fusion proteins (Table 6). Little staining of all Ig-fusion proteins was observed with LB33melA1 cells, a cell line that is hardly killed by NK cells (data not shown). All other cell lines that can be killed by NK cells were stained to various degrees with the Ig-fusion proteins (Table 6).

TABLE 6
Binding of different fusion proteins to melanoma cell lines

| Melanoma | CD99Ig | CD16Ig | NKp30Ig | NKp44Ig | NKp46Ig |
|---|---|---|---|---|---|
| cell lines | MFI | MFI | MFI | MFI | MFI |
| L33melA1 | 0.0 | 0.20 | 2.71 | 3.28 | 0.52 |
| L33me1B1 | 0.0 | 1.14 | 8.76 | 6.41 | 1.45 |
| 1106mel | 0.0 | 3.70 | 39.1 | 15.22 | 3.05 |
| FO-1 | 0.0 | 2.44 | 12.32 | 13.47 | 2.26 |
| 1259mel | 0.0 | 1.24 | 13.81 | 11.63 | 6.01 |
| 1047mel | 0.0 | 1.16 | 12.42 | 24.35 | 2.44 |
| 1612mH | 0.0 | 0.0 | 4.49 | 20.15 | 9.75 |
| 1612mel | 0.0 | 0.1 | 2.53 | 15.28 | 2.71 |

TABLE 7
Sequence listing

| | | amino acids/ |
|---|---|---|
| SEQ ID NO | Description | nucleotides |
| 1 | NKp46 cDNA (isoform a) | nucleotides |
| 2 | Fc Portion of IgG-cDNA | nucleotides |
| 3 | NKp46-Ig (isoform a) fusion | nucleotides |
| | protein-cDNA | |
| 4 | NKp46 (isoform a) | amino acids |
| 5 | Fc Portion of IgG | amino acids |
| 6 | NKp46-Ig (isoform a) fusion protein | amino acids |
| 7 | NKp44 cDNA | nucleotides |
| 8 | NKp44-Ig fusion protein cDNA | nucleotides |
| 9 | NKp44 | amino acids |
| 10 | NKp44-Ig fusion protein | amino acids |
| 11 | NKp46 cDNA (isoform b) | nucleotides |
| 12 | NKp46-Ig (isoform b) fusion | nucleotides |
| | protein-cDNA | |
| 13 | NKp46 (isoform b) | amino acids |
| 14 | NKp46-Ig (isoform b) fusion protein | amino acids |
| 15 | NKp30 cDNA | nucleotides |
| 16 | NKp30-Ig fusion protein-cDNA | nucleotides |
| 17 | NKp30 | amino acids |
| 18 | NKp30-Ig fusion protein | amino acids |
| 19 | NKp46D2 (isoform a) | nucleotides |
| 20 | NKp46D2 (isoform b) | nucleotides |
| 21 | NKp44D2 | nucleotides |
| 22 | NKp46D2 (isoform a) | amino acids |
| 23 | NKp46D2 (isoform b) | amino acids |
| 24 | NKp44D2 | amino acids |
| 25 | NKp46D1 (isoform a) | nucleotides |
| 26 | NKp46D1 (isoform a) | amino acids |

1. A targeting complex, capable of targeting an active substance to a target cell, said complex comprising:
a. a target recognition segment comprising one of NKp46, NKp30, NKp44 or a functional fragment thereof; and
b. an active segment comprising said active substance, said active substance being selected from the group consisting of:
a cytotoxic moiety;
an imaging moiety; and
an Ig fragment.

2. The complex of claim 1, wherein said NKp46 fragment comprises at least one of domains 1 and 2 of the NKp46 molecule.

3. The complex of claim 2, wherein said NKp46 fragment comprises domains 1 and 2 of the NKp46 molecule and has the amino acid sequence substantially as denoted by any one of SEQ ID NO: 4 and SEQ ID NO: 13.

4. The complex of claim 2, wherein said NKp46 fragment comprises domain 2 of the NKp46 molecule and has the amino acid sequence substantially as denoted by any one of SEQ ID NO: 22 and SEQ ID NO: 23.

5. The complex of claim 1, wherein said NKp44 fragment comprises at least one of domains 1 and 2 of the NKp44 molecule.

6. The complex of claim 5, wherein said NKp44 fragment comprises domains 1 and 2 of the NKp44 molecule and has the amino acid sequence substantially as denoted by SEQ ID NO: 9.

7. The complex of claim 5, wherein said NKp44 fragment comprises domain 2 of the NKp44 molecule and has the amino acid sequence substantially as denoted by SEQ ID NO: 24.

8. The complex of any one of claims 1 to 7, wherein said complex is a fusion protein comprising as said active segment an Ig fragment, and said Ig fragment is the Fc portion of an Ig molecule, encoded by the amino acid sequence substantially as denoted by SEQ ID NO: 5.

9. The complex of any of claims 1 to 7, wherein said complex is a conjugate comprising as said active segment a cytotoxic moiety, selected from cytotoxins or anticellular agents capable of killing and/or suppressing the growth or cell division of said target cell.

10. The conjugate of claim 9, wherein said cytotoxin or anticellular agent is any one of synthetic and plant-, fungus-, or bacteria-derived toxin.

11. The conjugate of claim 10 wherein said toxin is selected from the group consisting of A chain toxin, ribosome inactivating protein, α-sarcin, aspergillin, restrictocin, ribonuclease, diphtheria toxin, Pseudomonas exotoxin, an endotoxin or the lipid A moiety of an endotoxin.

12. The complex of any of claims 1 to 7, wherein said complex is a conjugate comprising as a said active segment an imaging moiety, which imaging moiety is selected from the group consisting of paramagnetic, radioactive and fluorogenic agents.

13. The complex of claim 1, wherein said target cell is a diseased cell.

14. The complex of claim 13, wherein said diseased cell is a cancer cell.

15. The complex of claim 14, wherein said cancer cell is selected from the group consisting of carcinomas, melanomas, lymphomas and sarcomas.

16. The complex of claim 1, wherein said target cell is a pathogenic virus-infected cell.

17. The complex of claim 16, wherein said pathogenic virus is any one of Influenza virus, human immunodeficiency virus, Epstein-Barr virus, cytomegalovirus, Vaccinia virus, MVM, ECMV and Herpes virus.

18. The complex of claim 17, wherein said target recognition segment is capable of binding to a ligand expressed on the surface of said target cell, said binding being sialic acid mediated.

19. An expression vector comprising a DNA coding for a NKp46-Ig fusion protein, said protein comprising:
a. a target recognition segment which comprises NKp46 or functional fragments thereof; and
b. an active segment which is the Fc portion of an Ig molecule.

20. The expression vector of claim 19, wherein said NKp46 fragment comprises at least one of domains 1 and 2 of the NKp46 molecule.

21. The expression vector of claim 20, wherein said NKp46 fragment comprises domains 1 and 2 of the NKp46 molecule encoded by the nucleic acid sequence substantially as denoted by any one of SEQ ID NO: 1 and SEQ ID NO: 11 and the Fc portion of an Ig molecule is encoded by the nucleic acid sequence substantially as denoted by SEQ ID NO: 2.

22. The expression vector of claim 20, wherein said NKp46 fragment comprises domain 2 of the NKp46 molecule encoded by the nucleic acid sequence substantially as denoted by any one of SEQ ID NO: 19 and SEQ ID NO: 20 and the Fc portion of an Ig molecule is encoded by the nucleic acid sequence substantially as denoted by SEQ ID NO: 2.

23. An expression vector comprising a DNA coding for a NKp44-Ig fusion protein said protein comprising:
a. a target recognition segment which comprises NKp44 or functional fragments thereof; and
b. an active segment which is the Fc portion of an Ig molecule.

24. The expression vector of claim 23, wherein said NKp44 fragment comprises at least one of domains 1 and 2 of the NKp44 molecule.

25. The expression vector of claim 24, wherein said NKp44 fragment comprises domains 1 and 2 of the NKp44 molecule encoded by the nucleic acid sequence substantially as denoted by SEQ ID NO: 7 and the Fc portion of an Ig molecule is encoded by the nucleic acid sequence substantially as denoted by SEQ ID NO: 2.

26. The expression vector of claim 24, wherein said NKp44 fragment comprises domain 2 of the NKp44 molecule encoded by the nucleic acid sequence substantially as denoted by SEQ ID NO: 21 and the Fc portion of an Ig molecule is encoded by the nucleic acid sequence substantially as denoted by SEQ ID NO: 2.

27. A host cell transformed with the expression vector of any one of claims 21 and 22.

28. A host cell transformed with the expression vector of any one of claims 25 and 26.

29. A NKp46-Ig fusion protein comprising the amino acid sequence substantially as denoted by any one of SEQ ID NO: 6, SEQ ID NO: 14, encoded by the nucleic acid sequence substantially as denoted by SEQ ID NO: 3 and SEQ ID NO: 12, respectively.

30. A NKp44-Ig fusion protein comprising the amino acid sequence substantially as denoted by SEQ ID NO: 10, encoded by the nucleic acid sequence substantially as denoted by SEQ ID NO: 8.

31. An antibody that specifically recognizes and binds to the fusion protein of claim 29.

32. An antibody that specifically recognizes and binds to the fusion protein of claim 30.

33. An antibody that specifically recognizes and binds to an epitope on a protein, wherein said protein is a ligand for any one of the NK cell activating receptor NKp46 and NKp44.

34. An antibody according to claim 33, designated as 135.7, wherein said ligand is a protein having the molecular weight of approximately 70 Kd.

35. An antibody according to any one of claims 31 to 34, selected from the group consisting of monoclonal and polyclonal antibodies.

36. An antibody according to claim 35, conjugated to a detectable moiety.

37. A composition for the treatment of a pathological condition comprising as active ingredient a complex comprising:
a. a target recognition segment capable of specifically recognizing and binding to a diseased target cell involved with said pathological condition, said recognition segment comprising one of NKp46, NKp44, NKp30 or a biologically functional fragment thereof; and
b. an active segment selected from the group consisting of a cytotoxic moiety and an Ig fragment.

38. The composition of claim 37, wherein said pathological condition is a cancer disease selected from the group comprising of carcinomas, melanomas, lymphomas and sarcomas.

39. The composition of claim 37, wherein said pathological condition is a viral infection caused by any one of Influenza virus, human immunodeficiency virus, Epstein-Barr virus, cytomegalovirus, Vaccinia virus, MVM, ECMV and Herpes virus.

40. The composition of claim 39, wherein the target recognition segment comprising NKp46, NKp44 or functional fragments thereof is capable of binding to a ligand expressed on the surface of said target cell, said binding being sialic acid mediated.

41. The composition of any one of claims 37 to 40, wherein said NKp46 fragment comprises at least one of domains 1 and 2 of the NKp46 molecule.

42. The composition of claim 41, wherein said NKp46 fragment comprises domains 1 and 2 of the NKp46 molecule and has the amino acid sequence substantially as denoted by any one of SEQ ID NO: 4 and SEQ ID NO: 13.

43. The composition of claim 41, wherein said NKp46 fragment comprises domain 2 of the NKp46 molecule and has the amino acid sequence substantially as denoted by any one of SEQ ID NO: 22 and SEQ ID NO: 23.

44. The composition of any one of claims 37 to 40, wherein said NKp44 fragment, comprises at least one of domains 1 and 2 of the NKp44 molecule.

45. The composition of claim 44, wherein said NKp44 fragment comprises domains 1 and 2 of the NKp44 molecule and has the amino acid sequence substantially as denoted by SEQ ID NO: 9.

46. The composition of claim 44, wherein said NKp44 fragment comprises domain 2 of the NKp44 molecule and has the amino acid sequence substantially as denoted by SEQ ID NO: 24.

47. The composition of any one of claims 42, 43, 45 and 46, wherein said active segment is an Ig fragment which is the Fc portion of an Ig molecule encoded by the amino acid sequence substantially as denoted by SEQ ID NO: 5.

48. The composition of any one of claims 42, 43, 45 and 46, wherein said active segment is a cytotoxic moiety, selected from cytotoxins or anticellular agents capable of killing and/or suppressing the growth and/or cell division of said target cell.

49. The composition of claim 48, wherein said cytotoxin or anticellular agent is any one of synthetic and plant-, fungus-, or bacterium-derived toxin.

50. The composition of claim 49, wherein said toxin is selected from the group consisting of A chain toxin, ribosome inactivating protein, α-sarcin, aspergillin, restrictocin, ribonuclease, diphtheria toxin, Pseudomonas exotoxin, an endotoxin or the lipid A moiety of an endotoxin.

51. A diagnostic composition for detecting the presence of diseased cells in a sample comprising a complex comprising:
a. a target recognition segment capable of specifically recognizing and binding to a diseased target cell involved with said pathological condition, said recognition segment comprising one of NKp46, NKp44, NKp30 or a biologically functional fragment thereof; and
b. a detectable imaging segment which is an imaging moiety selected from the group consisting of paramagnetic, radioactive and fluorogenic agent.

52. The diagnostic composition of claim 51, wherein said NKp46 fragment comprises at least one of domains 1 and 2 of the NKp46 molecule.

53. The diagnostic composition of claim 52, wherein said NKp46 fragment comprises domains 1 and 2 of the NKp46 molecule and has the amino acid sequence substantially as denoted by any one of SEQ ID NO: 4 and SEQ ID NO: 13.

54. The diagnostic composition of claim 52, wherein said NKp46 fragment comprises domain 2 of the NKp46 molecule and has the amino acid sequence substantially as denoted by any one of SEQ ID NO: 22 and SEQ ID NO: 23.

55. The diagnostic composition of claim 51, wherein said NKp44 fragment comprises at least one of domains 1 and 2 of the NKp44 molecule.

56. The diagnostic composition of claim 55, wherein said NKp44 fragment comprises domains 1 and 2 of the NKp44 molecule and has the amino acid sequence substantially as denoted by SEQ ID NO: 9.

57. The diagnostic composition of claim 55, wherein said NKp44 fragment comprises domain 2 of the NKp44 molecule and has the amino acid sequence substantially as denoted by SEQ ID NO: 24.

58. The diagnostic composition of any one of claims 51 to 57, wherein the target recognition segment is capable of binding to a ligand expressed on the surface of said target cell.

59. The diagnostic composition of claim 58, wherein said target cell is a cancer cell.

60. The diagnostic composition of claim 59, wherein said cancer is a cancer disease selected from the group consisting of carcinomas, melanomas, lymphomas and sarcomas.

61. A method for treating a pathological condition in a subject comprising administering to the subject a therapeutically effective amount of a complex or pharmaceutical agent comprising the same, said complex comprising:
a. a first target recognition segment capable of specifically recognizing and binding to a diseased target cell involved with said pathological condition, said recognition segment comprising one of NKp46, NKp44, NKp30 or a biologically functional fragment thereof; and
b. an active segment selected from the group consisting of a cytotoxic moiety and an Ig fragment.

62. The method of claim 61, wherein said pathological condition is a cancer disease selected from the group consisting of carcinomas, melanomas, lymphomas and sarcomas.

63. The method of claim 61, wherein said pathological condition is a viral infection caused by any one of Influenza virus, human immunodeficiency virus, Epstein-Barr virus, cytomegalovirus, Vaccinia virus, MVM, ECMV and Herpes virus.

64. The method of claim 63, wherein the target recognition segment NKp46, NKp44 or functional fragments thereof is capable of binding to a ligand expressed on the surface of said target cell, said binding being sialic acid mediated.

65. The method according to any one of claims 61 to 64, wherein said NKp46 fragment comprises at least one of domains 1 and 2 of the NKp46 molecule.

66. The method according to claim 65, wherein said NKp46 fragment comprises domains 1 and 2 of the NKp46 molecule and has the amino acid sequence substantially as denoted by any one of SEQ ID NO: 4 and SEQ ID NO: 13.

67. The method according to claim 65, wherein said NKp46 fragment comprises domain 2 of the NKp46 molecule and has the amino acid sequence substantially as denoted by any one of SEQ ID NO: 22 and SEQ ID NO: 23.

68. The method according to any one of claims 61 to 64, wherein said NKp44 fragment comprises at least one of domains 1 and 2 of the NKp44 molecule.

69. The method according to claim 68, wherein said NKp44 fragment, comprises domains 1 and 2 of the NKp44 molecule and has the amino acid sequence substantially as denoted by SEQ ID NO: 9.

70. The method according to claim 68, wherein said NKp44 fragment, comprises domain 2 of the NKp44 molecule and has the amino acid sequence substantially as denoted by SEQ ID NO: 24.

71. The method of any one of claims 66, 67, 69 and 70, wherein said active segment is an Ig fragment which is the Fc portion of an Ig molecule encoded by the amino acid sequence substantially as denoted by SEQ ID NO: 5.

72. The method of any one of claims 66, 67, 69 and 70, wherein said active segment is a cytotoxic moiety, selected from cytotoxins or anticellular agents capable of killing and/or suppressing the growth and/or cell division of said target cell.

73. The method of claim 72, wherein said cytotoxin or anticellular agent is any one of synthetic, and plant-, fungus-, or bacterium-derived toxin.

74. The method of claim 73, wherein said toxin is selected from the group consisting of A chain toxin, ribosome inactivating protein, α-sarcin, aspergillin, restrictocin, ribonuclease, diphtheria toxin, Pseudomonas exotoxin, an endotoxin or the lipid A moiety of an endotoxin.

75. A method for the diagnosis and imaging of malignant cells in a subject comprising the steps of introducing an imaging agent into the blood-stream of said subject, and detecting and quantitating the binding of said imaging agent to any one of NKp46, NKp30 and NKp44 ligands expressed on target malignant cells, wherein said imaging agent comprises a complex having:
a. a first target recognition segment, capable of specifically recognizing and binding to said malignant cells, said recognition segment comprising one of NKp46, NKp30, NKp44 or a biologically functional fragment thereof; and
b. an active segment which is an imaging moiety, said imaging moiety being selected from the group consisting of paramagnetic, radioactive and fluorogenic agents.

76. The method of claim 75, wherein said NKp46 fragment comprises at least one of domains 1 and 2 of the NKp46 molecule.

77. The method of claim 76, wherein said NKp46 fragment comprises domains 1 and 2 of the NKp46 molecule and has the amino acid sequence substantially as denoted by any one of SEQ ID NO: 4 and SEQ ID NO: 13.

78. The method of claim 76, wherein said NKp46 fragment comprises domain 2 of the NKp46 molecule and has the amino acid sequence substantially as denoted by any one of SEQ ID NO: 22 and SEQ ID NO: 23.

79. The method of claim 75, wherein said NKp44 fragment comprises at least one of domains 1 and 2 of the NKp44 molecule.

80. The method of claim 79, wherein said NKp44 fragment comprises domains 1 and 2 of the NKp44 molecule and has the amino acid sequence substantially as denoted by SEQ ID NO: 9.

81. The method of claim 79, wherein said NKp44 fragment comprises domain 2 of the NKp44 molecule and has the amino acid sequence substantially as denoted by SEQ ID NO: 24.

82. The method of any one of claims 75 to 81, wherein said malignant cells are selected from the selected from the group consisting of carcinomas, melanomas, lymphomas and sarcomas.
